SEQUENCE LISTING (30 sequences)

SEQ ID NO: 1 - DNA, 1941 bp, Elizabethkingia meningoseptica
  source 1..1941: mol_type = genomic DNA
atgaacccaataacttcaaaatttgacaaagtacttaatgcttcttccgaatacggacatgtaaaccatgaaccggattccagtaaagaacagcaacgaaacaccccgcaaaaatcaatgcccttttctgatcagattggaaattatcagagaaacaaagggattcctgtacaatcatatgacaatagtaagatttacattataggcagtggaatcgcaggtatgtcggcagcttattattttatacgcgatgggcatgttcctgcaaaaaacatcaccttcttggaacaattgcatatcgatggcggttcattagatggtgccggaaatccgacagacggctatattatccgtggcggtcgtgaaatggacatgacgtacgaaaatctttgggatatgtttcaggatatacctgccttagaaatgcctgctccttacagtgtactggacgaatacagattaattaatgataacgactccaattattctaaagcccggttaatcaacaataaaggtgagataaaagactttagcaagttcggcctaaataaaatggaccagttagctattatcagattacttctgaaaaataaagaagaactggacgatttaaccattgaggattacttcagcgaatccttcctgaaaagtaatttctggactttttggagaacgatgtttgcctttgaaaactggcatagcttattggaactgaaactttacatgcaccgtttccttcacgccatagacggactgaacgatctgtcttcactggtattccctaaatacaaccaatacgacaccttcgtaactcctctgcgcaaattccttcaggaaaaaggtgttaatatccacctgaacactctggtaaaagatctggatatccacatcaataccgaaggaaaagttgtagaaggaattatcaccgaacaggatggtaaggaagtaaaaatccctgttggtaaaaatgactatgtcattgtaactacaggttccatgacggaagataccttctacggaaataataaaactgctcctattattggcatagacaacagcacaagcggacaaagtgccggatggaagttgtggaaaaatctggctgcaaaatcagaaatttttgggaaaccagagaaattctgcagcaatatcgagaaatctgcatgggaatctgcaacgctaacctgtaaaccttcagcccttatcgacaagctgaaagaatactctgttaacgatccatattccggaaaaactgttaccggcggtattattaccattacagattccaactggctgatgagtttcacctgcaacagacagccacacttcccggaacagccggatgatgtactggtactttgggtatatgccttattcatggacaaagagggaaactatatcaaaaaaacaatgctggaatgtacaggagatgaaattcttgcagaattatgctaccatttaggtattgaagatcagctggaaaatgtacagaaaaatacaattgtaagaactgcattcatgccctatataacttctatgtttatgccaagagctaaaggcgatcgccctagagtagtgcctgaaggctgtaaaaatctgggactggtaggtcagtttgtagaaaccaataatgatgtggtatttacaatggaaagctctgtaagaacagcgagaattgctgtctacaaattactaaacctcaacaaacaggttcctgatatcaatcctttacagtatgatatccgacatctgctaaaagcagcaaaaacactgaatgatgacaaaccatttgtaggtgaaggcttgttgagaaaagtccttaaaggaacttactttgaacatgtgttacctgccggtgcagcagaggaagaagaacatgaatcctttatcgctgaacatgtaaataagttcagagaatgggtaaaaggaataagaggataa

SEQ ID NO: 2 - DNA, 1194 bp, Castellaniella defragrans
  source 1..1194: mol_type = genomic DNA
atgcggttcacattgaagacgacggcgattgtgtcggccgccgccctgctggccggtttcgggccgccgccccgcgcggcggaactgccgccggggcggctcgccaccaccgaggactatttcgcgcagcaggcgaagcaggccgtcacccccgacgtgatggcccagctggcctacatgaactacatcgatttcatctcgcccttctacagccggggctgctccttcgaggcctgggagctcaagcacacgccgcagcgggtcatcaagtattcgatcgccttctatgcgtatggcctggccagcgtggcgctcatcgacccgaagctgcgtgcgctcgccggccatgacctggacatcgcggtctccaagatgaagtgcaagcgggtctggggcgactgggaggaagacgggttcggcaccgacccgatcgagaaagagaacatcatgtacaagggccacctgaacctgatgtacggcctctatcagctggtgaccggcagccgccggtacgaagccgagcatgcccacctcacccgcatcatccatgacgagatcgcggccaacccctttgccggcatcgtctgcgagccggacaattattttgtccagtgcaattcggtcgcctacctgagcctgtgggtctatgaccggctgcatggcaccgactaccgggcggccaccagggcctggctggatttcatccagaaggacctgatcgatcccgagcggggcgccttctacctgtcctatcaccccgagtccggcgcggtgaagccgtggatctcggcgtatacgacagcctggacgctcgccatggtgcacggcatggaccccgccttttccgagcgctactacccccggttcaagcagaccttcgtcgaggtctacgacgagggccgcaaggcccgggtgcgcgagacggccggcacggacgacgcggatggcggggtgggcctggcttcggcgttcaccctgctgctggcccgcgagatgggcgaccagcagctcttcgaccaattgctgaatcacctggagccgccggccaagccgagcatcgtctcggcctcgctgcggtacgagcatcccggcagcctgctgttcgacgagctgctgttcctcgccaaggtgcatgccggctttggcgccctgcttcggatgccgcctccggcggccaagctcgcagggaaataa

SEQ ID NO: 3 - DNA, 1941 bp, Saccharomyces cerevisiae
  source 1..1941: mol_type = other DNA; mol_type = other DNA; note = oleate hydratase codon optimized for Saccharomyces cerevisiae expression
atgaacccaatcacatctaaattcgacaaagttttgaacgcctcctctgaatatggtcacgttaatcatgaaccagactcttccaaggaacaacaacgtaacactccacaaaagtccatgccattctccgatcaaattggtaattaccaaagaaacaagggtatccctgtccaatcctacgacaactctaaaatctacattatcggttctggtattgctggtatgtctgccgcttattacttcatcagagatggtcatgttcctgctaagaacattaccttcttggaacaattgcacattgatggcggttccttggacggtgctggcaacccaaccgacggttacatcattagaggcggtagagaaatggacatgacttacgaaaacttgtgggacatgttccaagacattccagctttagaaatgccagccccatactccgtcttggatgaatacagattgatcaacgataacgattctaactactctaaagctagattgattaataacaaaggtgaaatcaaggatttctccaagttcggtttgaacaagatggaccaattggccatcattagattactattgaagaacaaggaggaattagatgacttgactatcgaagattatttttctgaatcttttttgaagtccaacttctggacattttggagaactatgttcgctttcgaaaactggcattctttgttagaattaaaattgtacatgcatagattcttgcacgccatcgatggtttgaacgacttgtcctcattagttttccctaagtacaaccaatacgacactttcgttactccattgagaaaattcttgcaagaaaagggtgtcaacattcacttgaatactttggttaaggacttggatattcacattaatactgagggtaaggtcgttgaaggtatcattactgaacaagacggtaaggaagtcaaaattcctgttggtaagaacgactacgttatcgttaccactggttctatgactgaagacactttctacggtaataacaaaaccgccccaatcattggtattgataattccacctctggtcaatccgctggttggaaattgtggaagaatttggccgctaagtcagaaatcttcggtaagccagaaaagttctgttctaacatcgaaaagtccgcttgggaatctgctaccttgacttgtaaaccatccgccttgatcgacaagctaaaagaatattctgtcaacgatccatactccggtaagactgttactggcggtattatcaccatcaccgactctaactggttaatgtccttcacctgcaacagacaaccacacttcccagaacaaccagatgacgtcttggttttgtgggtctacgctttgttcatggacaaggaaggtaactatattaaaaagactatgctagaatgtaccggtgacgaaattttagctgaattgtgctaccatttgggtatcgaagatcaattggaaaacgttcaaaagaacaccattgtcagaaccgccttcatgccatacatcacttctatgtttatgcctagagccaagggtgatcgtcctagagttgtcccagaaggttgcaaaaacttgggtctagtcggtcaatttgttgaaaccaataacgacgtcgttttcactatggaatcttccgttcgtaccgctagaattgccgtctacaagttattgaacttgaacaagcaagttccagatattaaccctttacaatacgacattagacacctattgaaggccgctaagactttgaacgatgacaagccatttgttggtgaaggtctattaagaaaagttttgaaaggtacctacttcgaacatgttttgccagctggtgccgctgaggaagaggaacacgaatccttcattgctgaacacgtcaacaagtttagagaatgggttaagggtattagaggttga

SEQ ID NO: 4 - DNA, 1194 bp, Saccharomyces cerevisiae
  source 1..1194: mol_type = other DNA; note = linalool dehydratase codon optimized for Saccharomyces cerevisiae expression
atgagatttaccttaaagacaactgctattgtttctgctgccgctttattggctggtttcggtccacctccacgtgccgctgaattgcctccaggtagattggctactaccgaagactatttcgctcaacaagccaagcaagctgttaccccagacgttatggctcaattggcttacatgaattacatcgacttcatttcaccattctattccagaggttgttctttcgaagcttgggaattgaagcatacaccacaacgtgttatcaagtactccattgctttctacgcttacggtttggcttccgttgctttaatcgacccaaagttgagagctttagctggtcacgacttagatatcgccgtttccaagatgaagtgtaaaagagtctggggtgattgggaggaagatggtttcggtactgatccaatcgaaaaggaaaacattatgtataagggtcacttgaacttgatgtacggtttgtaccaattggttaccggttctcgtagatacgaagctgaacatgcccacttaactcgtatcattcacgacgaaattgccgctaacccattcgccggtatcgtctgtgaaccagataactatttcgttcaatgtaactctgttgcttacttgtctttgtgggtttacgacagattgcacggtaccgattacagagccgctactagagcttggttagacttcatccaaaaggacttgattgacccagaaagaggtgctttttacttgtcttaccaccctgaatctggtgctgtcaagccatggatctcagcttacactaccgcttggactttggctatggttcacggtatggacccagctttttctgagcgttattacccaagattcaagcaaacattcgtcgaagtttacgacgaaggtagaaaggctagagtccgtgaaaccgctggtaccgatgacgctgacggcggtgttggtttggcctccgctttcactttgttattggccagagaaatgggtgaccaacaactattcgatcaattattgaaccacttagaacctccagctaagccatctattgtttccgcttcattaagatatgaacacccaggttcattgctattcgacgaattattgttcctagctaaggttcacgctggtttcggtgctttgttaagaatgccacctccagctgccaaattagctggtaagtga

SEQ ID NO: 5 - DNA, 1221 bp, Rubrivivax gelatinosus
  source 1..1221: mol_type = genomic DNA
atgcgagcagcggagtctggcgctgatgcgcgagtgcggcctgtcgatcgagtggagcccgcagacgcaccggctggtgacgccggcggacttcgagcggctgttcccggcgacggggggagcgctgtacggcccggcgacgcacggctggatgtcctcgttccaccgggcctcgtcgacgagccggctgccggggctttacctggcggggggcagcgtgcacccggggccgggcgtgccgatggcggcgatgtccggccggttggcggccgagacgctgatggcgcacctcgattcgaccaaccggtcccgccgggtggttatctctggtggtatgtcgacgcggtcagcgacgacgggcggcatggcctgaccttcatcgccttcgtcggcagcgtcttctcgccttattacgcgtgggccggcggcccgaaggccgaccgtgccgacccggagaaccactgcgcgctgaacatcgcgctgtacggtgacgccggcaagcgctggacgatgaccgaacgcggccggcgctggatgcggcgcagccgcgacgagttcgtcatcggcccgagccggctgcactgggacggcgagtcgctgctcgtcgagttcgacgaggtcggcgtgccgatcccgcggcgcgtgaagggccgggtgcgcgtctggccgaaggcgctgtgccgcttcgtcacctcgctggacagcggcggacgccatcgctgggggccgatcgcgccttgcagccgcatcgaggtcgaactcgacagcccgcgtgtgcgctggagcggccacgcctacctcgactcgaacgaaggcgacgagccgatcgaccgcccgttccgcgagtgggactggtcgcgcgccacgatggccgacagcagcaccgcggtgatctacgacgtgcgccagaagcgcgacggcgatcgcgtgatcgccgagcgtttcctgctcgacggcagcaccgagagcttcgaggcgccgccgcggcagccgctgccgacgacgctgtggcgcatcggccgcacgatgcgtaccgagcccggcgtgccggcgctcgtcgagcagacgctggaggacacgccgttctacgcgcgttcgatggtgcgttcggggctgctcggcgaggtcgtgacctcggtgcacgagacgatgctgctgccgcgcgtgatcacgctgccggtgcggctgatgctgccctggcgcatgccgcgccgcgcttga

SEQ ID NO: 6 - DNA, 939 bp, Deinococcus radiodurans
  source 1..939: mol_type = genomic DNA
ttgatctcgccccccctcctgcgctgggggctggccgccgctggcctcggcctcgcctttctgggggcgctgctggcgctggccgagcagtccgcaggctgggctctgattgcgctggggctgccgctctccggggtgtttgcgctggcgggggacgcgctggggtcagcgttcggggccaccctgcggcggcgctggcaacggctgctcgctgagacgccgccgtggttggggtggctcgcgctctccgtggccctcaagattccggtgccgctgtggccgcaggggtttgcgctgctggggctgctcagcaccggggcgctgttcgtcgccggactgagctacgccgcgcagcgggcgggctggggccaggccgcgcagctcgccgccctcgcctgcctcgccgggctgggcgtggaactgctcggcagccggacgggggttccttttgggcagtactcctacgccaccgcccccgcgcccaccgtgctcacggtgccgcttatcgtgccgctgggctggttcgcctttaccctcgccgggctgcaactcgcgggcgggcgcccctggctggcggggctgctcatcacgtgctgggacgtgggcctcgaaccgctgatgacggcgcagaactactggcgctggagcgacccccacccgctgtgggccggggcgccggtgcagaactttctcggctggtgggcggtgggcacggcgattgcctggggcatgaagcgtctggcgccggggctttttgatgccaaagagaagcagaaggctgagggcttatcgccgtccttctcgctcgcctacttcaccgaggctttttttctgccgggcgggctggtgctggtggggcggctggcggaagcgggggtcacactcgccgtgatggggctgggagcgctgctcgcgcgcggggtgcggcgtgccagttga

SEQ ID NO: 7 - DNA, 1941 bp, Paracoccus denitrificans
  source 1..1941: mol_type = other DNA; note = oleate hydratase optimized for Paracoccus denitrificans expression
atgaaccccatcacgtcgaaattcgacaaggtgctgaacgcgtcgagcgagtatgggcatgtgaaccacgagccggatagctccaaggagcaacagcgcaacacgccgcagaagtcgatgccgttctcggaccagatcgggaactatcagcgcaacaagggcatccccgtgcagtcctatgacaacagcaagatctacatcatcggctcggggatcgccggcatgtcggcggcctattacttcatccgcgacggccacgtgccggccaaaaacatcacgtttctcgagcagctgcatatcgacgggggctcgctggacggcgccggcaacccgaccgacggctatatcatccgcgggggccgcgagatggatatgacctatgagaatctgtgggatatgttccaggatatccccgcgctggaaatgccggcgccctattcggtcctggacgaatatcgcctgatcaacgacaacgacagcaactactccaaagcccggctgatcaacaataagggggagatcaaggacttctccaagttcggccttaacaagatggaccagctggccatcatccgcctgcttctgaagaacaaggaagagctggacgatctgacgatcgaggactatttctcggaatcctttctgaagtcgaacttttggaccttctggcggacgatgttcgccttcgaaaactggcatagccttctggagctgaagctgtatatgcaccggttcctgcatgccatcgacgggctgaacgacctgagctcgctggtgttcccgaagtacaaccagtacgatacgttcgtcaccccgcttcgcaaatttctgcaggagaagggcgtgaacatccacctgaacaccctggtgaaggatctggatatccatatcaacaccgaaggcaaggtcgtggagggcatcatcaccgaacaggacggcaaggaagtcaagatcccggtcggcaagaacgattatgtgatcgtcacgaccgggtcgatgaccgaagacaccttctatggcaataacaagacggccccgatcatcggcatcgacaactcgacctcgggccagtcggccggctggaagctgtggaagaacctggccgcgaagtccgagatcttcggcaagcccgagaagttctgctccaacatcgagaaatccgcgtgggagtcggcgacgcttacctgcaagccgagcgccctcatcgacaagctgaaagagtactcggtcaacgatccctactccggcaagacggtcaccgggggcatcatcacgatcaccgacagcaactggctgatgtcgttcacgtgcaatcgccagccccatttcccggaacaaccggatgacgtcctggtcctgtgggtgtatgccctgttcatggataaggagggcaactatatcaaaaagaccatgctcgagtgtacgggcgacgagatcctggcggagctgtgctatcacctgggcatcgaggaccagctggagaacgtgcaaaagaacacgatcgtccggaccgccttcatgccgtacatcacctcgatgtttatgccccgcgccaagggggatcgcccccgcgtggtccccgaaggctgcaaaaacctgggcctggtcggccagttcgtggaaaccaataacgatgtcgtgttcaccatggaatcgtccgtgcgcaccgcccggatcgccgtgtacaaactcctgaaccttaacaaacaagtgccggacatcaacccgctgcagtatgacatccgccacctgctcaaggccgcgaagaccctcaacgatgacaagcccttcgtgggggagggccttctgcggaaggtcctgaagggcacctattttgaacatgtgctgccggcgggcgccgcggaagaggaagagcatgagtcgttcatcgccgaacatgtcaataagtttcgcgaatgggtcaagggcatccgcggctaa

SEQ ID NO: 8 - DNA, 1194 bp, Paracoccus denitrificans
  source 1..1194: mol_type = other DNA; note = linalool dehydratase codon optimized for Paracoccus denitrificans expression
atgcgcttcaccctgaagaccacggcgatcgtgagcgcggccgcgcttctggccggcttcgggcccccgccccgcgcggccgagctgcccccgggccgcctcgccacgaccgaagactacttcgcccagcaagccaagcaggcggtgaccccggacgtcatggcgcagcttgcctatatgaactacatcgacttcatcagcccgttctactcgcgcggctgcagcttcgaggcgtgggaactgaagcatacgccccagcgggtcatcaaatattcgatcgcgttctacgcctacggcctggcgtcggtcgcgcttatcgatccgaagctgcgcgccctggccggccatgatctggacatcgccgtgtcgaagatgaaatgcaagcgcgtctggggcgactgggaggaagacggcttcgggacggatccgatcgaaaaggagaatatcatgtacaaagggcatctgaatctgatgtatggcctgtatcagctggtcacgggcagccgccggtatgaggccgagcatgcccatctgacccgcatcatccacgacgaaatcgcggccaaccccttcgccggcatcgtgtgcgagcccgataattatttcgtccaatgtaattcggtcgcctacctgtcgctgtgggtgtatgaccgcctgcatggcaccgattaccgcgcggccacccgcgcctggctggatttcatccagaaggacctgatcgacccggagcggggcgccttttaccttagctatcatccggagtccggggcggtcaaaccctggatctcggcgtacaccacggcgtggacgcttgccatggtccacggcatggatccggcgttcagcgagcgctactatccgcggtttaagcagacctttgtcgaggtgtatgacgagggccggaaagcccgcgtccgggagacggcgggcaccgatgacgcggacgggggcgtcggcctggcgtcggccttcacgcttctgcttgcccgcgaaatgggcgaccaacagctgttcgaccagcttctgaaccatctcgaacccccggcgaagccgtccatcgtgagcgccagcctgcgctatgagcatccggggtcgctcctgttcgacgagcttctgttcctcgccaaggtccatgccggcttcggcgccctgcttcgcatgccgcccccggcggccaagctggcgggcaaatga

SEQ ID NO: 9 - DNA, 1221 bp, Paracoccus denitrificans
  source 1..1221: mol_type = other DNA; note = acyclic carotenoid 1,2-hydratase codon optimized for Paracoccus denitrificans
atgcgcgcggccgaaagcggcgccgacgcccgcgtccgccccgtggatcgcgtggaacccgccgacgcccccgccggcgacgccgggggccttcgggccgcggtcccgggcgatgggggctccgcggtgcgcccgggggacgcgcgccttgacgtgctggtgccgcccggcctggtcgatgagccggcggccggggccctgccgggcgggggccaacgcgccccgggcgccggccgcgccgatgggggcgacgtgcgccccgtcgggggccgcgacgcggacggcgcgccgcgctttgatcaaccggtgccgcccgggggctacctgtggtggtatgtcgatgccgtctcggacgatgggcggcatggccttaccttcatcgcgttcgtcggctcggtcttctccccctactatgcctgggcgggcgggcccaaagccgaccgggccgaccccgaaaatcattgcgccctgaacatcgcgctgtatggggatgcgggcaagcgctggaccatgaccgagcgcggccggcgctggatgcggcgctcccgcgacgagttcgtgatcggcccctcgcgcctgcattgggatggggagtcgctcctggtggaattcgacgaggtcggggtgccgatcccgcggcgcgtcaaaggccgcgtgcgcgtctggccgaaggcgctctgccgctttgtcaccagcctggatagcgggggccgccatcgctggggccccatcgccccgtgctcgcgcatcgaggtcgagctggattcgccccgcgtgcgctggtccggccacgcgtatctggactcgaacgagggggacgagccgatcgatcgcccgttccgcgagtgggactggagccgcgccacgatggccgactcctcgaccgcggtgatctatgatgtgcgccagaagcgcgacggcgaccgggtgatcgccgagcgcttcctcctggatggctcgaccgagtcgtttgaggcgcccccgcgccaaccgcttcccacgaccctgtggcgcatcggccgcaccatgcgcaccgagccgggcgtgcccgccctggtggaacagacgctggaagatacccccttctatgcgcgcagcatggtgcggagcgggctgcttggcgaggtcgtgaccagcgtccatgaaaccatgctcctgccgcgcgtgatcaccctgccggtgcgcctgatgctcccgtggcgcatgccccgccgggcctga

SEQ ID NO: 10 - DNA, 939 bp, Paracoccus denitrificans
  source 1..939: mol_type = other DNA; note = carotenoid 1,2-hydratase CruF codon optimized for Paracoccus denitrificans
ctgatctcgcccccgctcctgcggtggggcctggcggccgcgggcctgggcctcgccttcctgggcgcgctcctggcgctggccgaacaatcggccgggtgggccctgatcgccctgggcctgccgctgtcgggcgtcttcgccctggccggcgatgcgctgggctccgcgtttggcgccacgctgcggcgccggtggcagcgccttctggccgagacgcccccgtggctgggctggctggccctgagcgtggcgctgaagatccccgtgccgctctggccccaaggcttcgcgcttctggggctcctgtcgaccggggccctcttcgtggccggcctgtcgtatgcggcccagcgggccggctggggccaggcggcccagcttgccgcgctggcgtgcctggccgggctgggggtcgagctcctgggctcccgcacgggcgtcccgttcgggcagtattcgtatgcgaccgccccggcccccaccgtcctgaccgtcccgctgatcgtcccgctgggctggtttgcctttaccctggcggggctgcagctggcgggcgggcgcccctggcttgccggccttctgatcacgtgctgggacgtgggcctggagccgctgatgaccgcccagaactattggcgctggtcggacccccatcccctgtgggccggcgccccggtccagaactttctgggctggtgggcggtgggcaccgccatcgcctgggggatgaagcgcctcgcccccggcctgttcgacgccaaggagaaacagaaggcggaaggcctgagcccgagcttctcgctggcgtacttcacggaggccttctttctgccgggcgggctggtgctggtgggccggctggccgaggccggcgtcaccctggcggtcatgggcctgggcgccctcctggcccgcggggtccggcgcgcctcgtga

SEQ ID NO: 11 - DNA, 1191 bp, Nitrospira sp.
  source 1..1191: mol_type = other DNA; note = linalool dehydratase codon optimized for Nitrospira japonica expression
atgcggttcacgctgaagacgacggccatcgtgtccgccgccgccctgctggccggcttcggcccgccgccgcgggccgccgaactgccgccgggccggctggccacgacggaagactatttcgcccagcaggccaagcaggccgtgacgccggacgtgatggcccagctggcctatatgaactatatcgacttcatctccccgttctattcccggggctgctccttcgaagcctgggaactgaagcatacgccgcagcgggtgatcaagtattccatcgccttctatgcctatggcctggcctccgtggccctgatcgacccgaagctgcgggccctggccggccatgacctggacatcgccgtgtccaagatgaagtgcaagcgggtgtggggcgactgggaagaagacggcttcggcacggacccgatcgaaaaggaaaacatcatgtataagggccatctgaacctgatgtatggcctgtatcagctggtgacgggctcccggcggtatgaagccgaacatgcccatctgacgcggatcatccatgacgaaatcgccgccaacccgttcgccggcatcgtgtgcgaaccggacaactatttcgtgcagtgcaactccgtggcctatctgtccctgtgggtgtatgaccggctgcatggcacggactatcgggccgccacgcgggcctggctggacttcatccagaaggacctgatcgacccggaacggggcgccttctatctgtcctatcatccggaatccggcgccgtgaagccgtggatctccgcctatacgacggcctggacgctggccatggtgcatggcatggacccggccttctccgaacggtattatccgcggttcaagcagacgttcgtggaagtgtatgacgaaggccggaaggcccgggtgcgggaaacggccggcacggacgacgccgacggcggcgtgggcctggcctccgccttcacgctgctgctggcccgggaaatgggcgaccagcagctgttcgaccagctgctgaaccatctggaaccgccggccaagccgtccatcgtgtccgcctccctgcggtatgaacatccgggctccctgctgttcgacgaactgctgttcctggccaaggtgcatgccggcttcggcgccctgctgcggatgccgccgccggccgccaagctggccggcaag

SEQ ID NO: 12 - DNA, 1937 bp, Nitrospira sp.
  source 1..1937: mol_type = other DNA; note = oleate hydratase codon optimized for Nitrospira japonica expression
atgaacccgatcacgtccaagttcgacaaggtgctgaacgcctcctccgaatatggccatgtgaaccatgaaccggactcctccaaggaacagcagcggaacacgccgcagaagtccatgccgttctccgaccagatcggcaactatcagcggaacaagggcatcccggtgcagtcctatgacaactccaagatctatatcatcggctccggcatcgccggcatgtccgccgcctattatttcatccgggacggccatgtgccggccaagaacatcacgttcctggaacagctgcatatcgacggcggctccctggacggcgccggcaacccgacggacggctatatcatccggggcggccgggaaatggacatgacgtatgaaaacctgtgggacatgttccaggacatcccggccctggaaatgccggccccgtattccgtgctggacgaatatcggctgatcaacgacaacgactccaactattccaaggcccggctgatcaacaacaagggcgaaatcaaggacttctccaattcggcctgaacaagatggaccagctggccatcatccggctgctgctgaagaacaaggaagaactggacgacctgacgatcgaagactatttctccgaatccttcctgaagtccaacttctggacgttctggcggacgatgttcgccttcgaaaactggcattccctgctggaactgaagctgtatatgcatcggttcctgcatgccatcgacggcctgaacgacctgtcctccctggtgttcccgaagtataaccagtatgacacgttcgtgacgccgctgcggaagttcctgcaggaaaagggcgtgaacatccatctgaacacgctggtgaaggacctggacatccatatcaacacggaaggcaaggtggtggaaggcatcatcacggaacaggacggcaaggaagtgaagatcccggtgggcaagaacgactatgtgatcgtgacgacgggctccatgacggaagacacgttctatggcaacaacaagacggccccgatcatcggcatcgacaactccacgtccggccagtccgccggctggaagctgtggaagaacctggccgccaagtccgaaatcttcggcaagccggaaaagttctgctccaacatcgaaaagtccgcctgggaatccgccacgctgacgtgcaagccgtccgccctgatcgacaagctgaaggaatattccgtgaacgacccgtattccggcaagacggtgacgggcggcatcatcacgatcacggactccaactggctgatgtccttcacgtgcaaccggcagccgcatttcccggaacagccggacgacgtgctggtgctgtgggtgtatgccctgttcatggacaaggaaggcaactatatcaagaagacgatgctggaatgcacgggcgacgaaatcctggccgaactgtgctatcatctgggcatcgaagaccagctggaaaacgtgcagaagaacacgatcgtgcggacggccttcatgccgtatatcacgtccatgttcatgccgcgggccaagggcgaccggccgcgggtggtgccggaaggctgcaagaacctgggcctggtgggccagttcgtggaaacgaacaacgacgtggtgttcacgatggaatcctccgtgcggacggcccggatcgccgtgtataagctgctgaacctgaacaagcaggtgccggacatcaacccgctgcagtatgacatccggcatctgctgaaggccgccaagacgctgaacgacgacaagccgttcgtgggcgaaggcctgctgcggaaggtgctgaagggcacgtatttcgaacatgtgctgccggccggcgccgccgaagaagaagaacatgaatccttcatcgccgaacatgtgaacaagttccgggaatgggtgaagggcatccggggc

SEQ ID NO: 13 - DNA, 936 bp, Nitrospira sp.
  source 1..936: mol_type = other DNA; note = carotenoid 1,2-hydratase (CruF) codon optimized for Nitrospira japonica expression
atgatctccccgccgctgctgcggtggggcctggccgccgccggcctgggcctggccttcctgggcgccctgctggccctggccgaacagtccgccggctgggccctgatcgccctgggcctgccgctgtccggcgtgttcgccctggccggcgacgccctgggctccgccttcggcgccacgctgcggcggcggtggcagcggctgctggccgaaacgccgccgtggctgggctggctggccctgtccgtggccctgaagatcccggtgccgctgtggccgcagggcttcgccctgctgggcctgctgtccacgggcgccctgttcgtggccggcctgtcctatgccgcccagcgggccggctggggccaggccgcccagctggccgccctggcctgcctggccggcctgggcgtggaactgctgggctcccggacgggcgtgccgttcggccagtattcctatgccacggccccggccccgacggtgctgacggtgccgctgatcgtgccgctgggctggttcgccttcacgctggccggcctgcagctggccggcggccggccgtggctggccggcctgctgatcacgtgctgggacgtgggcctggaaccgctgatgacggcccagaactattggcggtggtccgacccgcatccgctgtgggccggcgccccggtgcagaacttcctgggctggtgggccgtgggcacggccatcgcctggggcatgaagcggctggccccgggcctgttcgacgccaaggaaaagcagaaggccgaaggcctgtccccgtccttctccctggcctatttcacggaagccttcttcctgccgggcggcctggtgctggtgggccggctggccgaagccggcgtgacgctggccgtgatgggcctgggcgccctgctggcccggggcgtgcggcgggcctcc

SEQ ID NO: 14 - DNA, 1218 bp, Nitrospira sp.
  source 1..1218: mol_type = other DNA; note = carotenoid 1,2-hydratase (CrtC) codon optimized for Nitrospira japonica expression
atgcgggccgccgaatccggcgccgacgcccgggtgcggccggtggaccgggtggaaccggccgacgccccggccggcgacgccggcggcctgcgggccgccgtgccgggcgacggcggctccgccgtgcggccgggcgacgcccggctggacgtgctggtgccgccgggcctggtggacgaaccggccgccggcgccctgccgggcggcggccagcgggccccgggcgccggccgggccgacggcggcgacgtgcggccggtgggcggccgggacgccgacggcgccccgcggttcgaccagccggtgccgccgggcggctatctgtggtggtatgtggacgccgtgtccgacgacggccggcatggcctgacgttcatcgccttcgtgggctccgtgttctccccgtattatgcctgggccggcggcccgaaggccgaccgggccgacccggaaaaccattgcgccctgaacatcgccctgtatggcgacgccggcaagcggtggacgatgacggaacggggccggcggtggatgcggcggtcccgggacgaattcgtgatcggcccgtcccggctgcattgggacggcgaatccctgctggtggaattcgacgaagtgggcgtgccgatcccgcggcgggtgaagggccgggtgcgggtgtggccgaaggccctgtgccggttcgtgacgtccctggactccggcggccggcatcggtggggcccgatcgccccgtgctcccggatcgaagtggaactggactccccgcgggtgcggtggtccggccatgcctatctggactccaacgaaggcgacgaaccgatcgaccggccgttccgggaatgggactggtcccgggccacgatggccgactcctccacggccgtgatctatgacgtgcggcagaagcgggacggcgaccgggtgatcgccgaacggttcctgctggacggctccacggaatccttcgaagccccgccgcggcagccgctgccgacgacgctgtggcggatcggccggacgatgcggacggaaccgggcgtgccggccctggtggaacagacgctggaagacacgccgttctatgcccggtccatggtgcggtccggcctgctgggcgaagtggtgacgtccgtgcatgaaacgatgctgctgccgcgggtgatcacgctgccggtgcggctgatgctgccgtggcggatgccgcggcgggcc

SEQ ID NO: 15 - DNA, 1190 bp, Nitrobacter winogradskyi
  source 1..1190: mol_type = other DNA; note = linalool dehydratase codon optimized for Nitrobacter winogradskyi expression
atgcgcttcaccctgaagaccaccgcgatcgtctcggcggcggcgctgctggcgggcttcggcccgccgccgcgcgcggcggagctgccgccgggccgcctggcgaccaccgaggactatttcgcgcagcaggcgaagcaggcggtcaccccggacgtcatggcgcagctggcgtatatgaactatatcgacttcatctcgccgttctattcgcgcggctgctcgttcgaggcgtgggagctgaagcacaccccgcagcgcgtcatcaagtattcgatcgcgttctatgcgtatggcctggcgtcggtcgcgctgatcgacccgaagctgcgcgcgctggcgggccacgacctggacatcgcggtctcgaagatgaagtgcaagcgcgtctggggcgactgggaggaggacggcttcggcaccgacccgatcgagaaggagaacatcatgtataagggccacctgaacctgatgtatggcctgtatcagctggtcaccggctcgcgccgctatgaggcggagcacgcgcacctgacccgcatcatccacgacgagatcgcggcgaacccgttcgcgggcatcgtctgcgagccggacaactatttcgtccagtgcaactcggtcgcgtatctgtcgctgtgggtctatgaccgcctgcaggcaccgactatcgcgcggcgacccgcgcgtggctggacttcatccagaaggacctgatcgacccggagcgcggcgcgttctatctgtcgtatcacccggagtcgggcgcggtcaagccgtggatctcggcgtataccaccgcgtggaccctggcgatggtccacggcatggacccggcgttctcggagcgctattatccgcgcttcaagcagaccttcgtcgaggtctatgacgagggccgcaaggcgcgcgtccgcgagaccgcgggcaccgacgacgcggacggcggcgtcggcctggcgtcggcgttcaccctgctgctggcgcgcgagatgggcgaccagcagctgttcgaccagctgctgaaccacctggagccgccggcgaagccgtcgatcgtctcggcgtcgctgcgctatgagcacccgggctcgctgctgttcgacgagctgctgttcctggcgaaggtccacgcgggcttcggcgcgctgctgcgcatgccgccgccggcggcgaagctggcgggcaag

SEQ ID NO: 16 - DNA, 1937 bp, Nitrobacter winogradskyi
  source 1..1937: mol_type = other DNA; note = oleate hydratase codon optimized for Nitrobacter winogradskyi expression
atgaacccgatcacctcgaagttcgacaaggtcctgaacgcgtcgtcggagtatggccacgtcaaccacgagccggactcgtcgaaggagcagcagcgcaacaccccgcagaagtcgatgccgttctcggaccagatcggcaactatcagcgcaacaagggcatcccggtccagtcgtatgacaactcgaagatctatatcatcggctcgggcatcgcgggcatgtcggcggcgtattatttcatccgcgacggccacgtcccggcgaagaacatcaccttcctggagcagctgcacatcgacggcggctcgctggacggcgcgggcaacccgaccgacggctatatcatccgcggcggccgcgagatggacatgacctatgagaacctgtgggacatgttccaggacatcccggcgctggagatgccggcgccgtattcggtcctggacgagtatcgcctgatcaacgacaacgactcgaactattcgaaggcgcgcctgatcaacaacaagggcgagatcaaggacttctcgaagttcggcctgaacaagatggaccagctggcgatcatccgcctgctgctgaagaacaaggaggagctggacgacctgaccatcgaggactatttctcggagtcgttcctgaagtcgaacttctggaccttctggcgcaccatgttcgcgttcgagaactggcactcgctgctggagctgaagctgtatatgcaccgcttcctgcacgcgatcgacggcctgaacgacctgtcgtcgctggtcttcccgaagtataaccagtatgacaccttcgtcaccccgctgcgcaagttcctgcaggagaagggcgtcaacatccacctgaacaccctggtcaaggacctggacatccacatcaacaccgaggcaaggtcgtcgagggcatcatcaccgagcaggacggcaaggaggtcaagatcccggtcggcaagaacgactatgtcatcgtcaccaccggctcgatgaccgaggacaccttctatggcaacaacaagaccgcgccgatcatcggcatcgacaactcgacctcgggccagtcggcgggctggaagctgtggaagaacctggcggcgaagtcggagatcttcggcaagccggagaagttctgctcgaacatcgagaagtcggcgtgggagtcggcgaccctgacctgcaagccgtcggcgctgatcgacaagctgaaggagtattcggtcaacgacccgtattcgggcaagaccgtcaccggcggcatcatcaccatcaccgactcgaactggctgatgtcgttcacctgcaaccgccagccgcacttcccggagcagccggacgacgtcctggtcctgtgggtctatgcgctgttcatggacaaggagggcaactatatcaagaagaccatgctggagtgcaccggcgacgagatcctggcggagctgtgctatcacctgggcatcgaggaccagctggagaacgtccagaagaacaccatcgtccgcaccgcgttcatgccgtatatcacctcgatgttcatgccgcgcgcgaagggcgaccgcccgcgcgtcgtcccggagggctgcaagaacctgggcctggtcggccagttcgtcgagaccaacaacgacgtcgtcttcaccatggagtcgtcggtccgcaccgcgcgcatcgcggtctataagctgctgaacctgaacaagcaggtcccggacatcaacccgctgcagtatgacatccgccacctgctgaaggcggcgaagaccctgaacgacgacaagccgttcgtcggcgagggcctgctgcgcaaggtcctgaagggcacctatttcgagcacgtcctgccggcgggcgcggcggaggaggaggagcacgagtcgttcatcgcggagcacgtcaacaagttccgcgagtgggtcaagggcatccgcggc

SEQ ID NO: 17 - DNA, 936 bp, Nitrobacter winogradskyi
  source 1..936: mol_type = other DNA; note = carotenoid 1,2-hydratase (CruF) codon optimized for Nitrobacter winogradskyi expression
atgatctcgccgccgctgctgcgctggggcctggcggcggcgggcctgggcctggcgttcctgggcgcgctgctggcgctggcggagcagtcggcgggctgggcgctgatcgcgctgggcctgccgctgtcgggcgtcttcgcgctggcgggcgacgcgctgggctcggcgttcggcgcgaccctgcgccgccgctggcagcgcctgctggcggagaccccgccgtggctgggctggctggcgctgtcggtcgcgctgaagatcccggtcccgctgtggccgcagggcttcgcgctgctgggcctgctgtcgaccggcgcgctgttcgtcgcgggcctgtcgtatgcggcgcagcgcgcgggctggggccaggcggcgcagctggcggcgctggcgtgcctggcgggcctgggcgtcgagctgctgggctcgcgcaccggcgtcccgttcggccagtattcgtatgcgaccgcgccggcgccgaccgtcctgaccgtcccgctgatcgtcccgctgggctggttcgcgttcaccctggcgggcctgcagctggcgggcggccgcccgtggctggcgggcctgctgatcacctgctgggacgtcggcctggagccgctgatgaccgcgcagaactattggcgctggtcggacccgcacccgctgtgggcgggcgcgccggtccagaacttcctgggctggtgggcggtcggcaccgcgatcgcgtggggcatgaagcgcctggcgccgggcctgttcgacgcgaaggagaagcagaaggcggagggcctgtcgccgtcgttctcgctggcgtatttcaccgaggcgttcttcctgccgggcggcctggtcctggtcggccgcctggcggaggcgggcgtcaccctggcggtcatgggcctgggcgcgctgctggcgcgcggcgtccgccgcgcgtcg

SEQ ID NO: 18 - DNA, 1218 bp, Nitrobacter winogradskyi
  source 1..1218: mol_type = other DNA; note = carotenoid 1,2-hydratase (CrtC) codon optimized for Nitrobacter winogradskyi expression
atgcgcgcggcggagtcgggcgcggacgcgcgcgtccgcccggtcgaccgcgtcgagccggcggacgcgccggcgggcgacgcgggcggcctgcgcgcggcggtcccgggcgacggcggctcggcggtccgcccgggcgacgcgcgcctggacgtcctggtcccgccgggcctggtcgacgagccggcggcgggcgcgctgccgggcggcggccagcgcgcgccgggcgcgggccgcgcggacggcggcgacgtccgcccggtcggcggccgcgacgcggacggcgcgccgcgcttcgaccagccggtcccgccgggcggctatctgtggtggtatgtcgacgcggtctcggacgacggccgccacggcctgaccttcatcgcgttcgtcggctcggtcttctcgccgtattatgcgtgggcgggcggcccgaaggcggaccgcgcggacccggagaaccactgcgcgctgaacatcgcgctgtatggcgacgcgggcaagcgctggaccatgaccgagcgcggccgccgctggatgcgccgctcgcgcgacgagttcgtcatcggcccgtcgcgcctgcactgggacggcgagtcgctgctggtcgagttcgacgaggtcggcgtcccgatcccgcgccgcgtcaagggccgcgtccgcgtctggccgaaggcgctgtgccgcttcgtcacctcgctggactcgggcggccgccaccgctggggcccgatcgcgccgtgctcgcgcatcgaggtcgagctggactcgccgcgcgtccgctggtcgggccacgcgtatctggactcgaacgagggcgacgagccgatcgaccgcccgttccgcgagtgggactggtcgcgcgcgaccatggcggactcgtcgaccgcggtcatctatgacgtccgccagaagcgcgacggcgaccgcgtcatcgcggagcgcttcctgctggacggctcgaccgagtcgttcgaggcgccgccgcgccagccgctgccgaccaccctgtggcgcatcggccgcaccatgcgcaccgagccgggcgtcccggcgctggtcgagcagaccctggaggacaccccgttctatgcgcgctcgatggtccgctcgggcctgctgggcgaggtcgtcacctcggtccacgagaccatgctgctgccgcgcgtcatcaccctgccggtccgcctgatgctgccgtggcgcatgccgcgccgcgcg

SEQ ID NO: 19 - DNA, 1191 bp, Acidithiobacillus ferrooxidans
  source 1..1191: mol_type = other DNA; note = linalool dehydratase codon optimized for Acidithiobacillus ferrooxidans expression
atgcgcttcaccctgaagaccaccgccatcgtgagcgccgccgccctgctggccggcttcggcccgccgccgcgcgccgccgaactgccgccgggccgcctggccaccaccgaagattatttcgcccagcaggccaagcaggccgtgaccccggatgtgatggcccagctggcctatatgaattatatcgatttcatcagcccgttctatagccgcggctgcagcttcgaagcctgggaactgaagcataccccgcagcgcgtgatcaagtatagcatcgccttctatgcctatggcctggccagcgtggccctgatcgatccgaagctgcgcgccctggccggccatgatctggatatcgccgtgagcaagatgaagtgcaagcgcgtgtggggcgattgggaagaagatggcttcggcaccgatccgatcgaaaaggaaaatatcatgtataagggccatctgaatctgatgtatggcctgtatcagctggtgaccggcagccgccgctatgaagccgaacatgcccatctgacccgcatcatccatgatgaaatcgccgccaatccgttcgccggcatcgtgtgcgaaccggataattatttcgtgcagtgcaatagcgtggcctatctgagcctgtgggtgtatgatcgcctgcatggcaccgattatcgcgccgccacccgcgcctggctggatttcatccagaaggatctgatcgatccggaacgcggcgccttctatctgagctatcatccggaaagcggcgccgtgaagccgtggatcagcgcctataccaccgcctggaccctggccatggtgcatggcatggatccggccttcagcgaacgctattatccgcgcttcaagcagaccttcgtggaagtgtatgatgaaggccgcaaggcccgcgtgcgcgaaaccgccggcaccgatgatgccgatggcggcgtgggcctggccagcgccttcaccctgctgctggcccgcgaaatgggcgatcagcagctgttcgatcagctgctgaatcatctggaaccgccggccaagccgagcatcgtgagcgccagcctgcgctatgaacatccgggcagcctgctgttcgatgaactgctgttcctggccaaggtgcatgccggcttcggcgccctgctgcgcatgccgccgccggccgccaagctggccggcaag

SEQ ID NO: 20 - DNA, 1938 bp, Acidithiobacillus ferrooxidans
  source 1..1938: mol_type = other DNA; note = oleate hydratase codon optimized for Acidithiobacillus ferrooxidans expression
atgaatccgatcaccagcaagttcgataaggtgctgaatgccagcagcgaatatggccatgtgaatcatgaaccggatagcagcaaggaacagcagcgcaataccccgcagaagagcatgccgttcagcgatcagatcggcaattatcagcgcaataagggcatcccggtgcagagctatgataatagcaagatctatatcatcggcagcggcatcgccggcatgagcgccgcctattatttcatccgcgatggccatgtgccggccaagaatatcaccttcctggaacagctgcatatcgatggcggcagcctggatggcgccggcaatccgaccgatggctatatcatccgcggcggccgcgaaatggatatgacctatgaaaatctgtgggatatgttccaggatatcccggccctggaaatgccggccccgtatagcgtgctggatgaatatcgcctgatcaatgataatgatagcaattatagcaaggcccgcctgatcaataataagggcgaaatcaaggatttcagcaagttcggcctgaataagatggatcagctggccatcatccgcctgctgctgaagaataaggaagaactggatgatctgaccatcgaagattatttcagcgaaagcttcctgaagagcaatttctggaccttctggcgcaccatgttcgccttcgaaaattggcatagcctgctggaactgaagctgtatatgcatcgcttcctgcatgccatcgatggcctgaatgatctgagcagcctggtgttcccgaagtataatcagtatgataccttcgtgaccccgctgcgcaagttcctgcaggaaaagggcgtgaatatccatctgaataccctggtgaaggatctggatatccatatcaataccgaaggcaaggtggtggaaggcatcatcaccgaacaggatggcaaggaagtgaagatcccggtgggcaagaatgattatgtgatcgtgaccaccggcagcatgaccgaagataccttctatggcaataataagaccgccccgatcatcggcatcgataatagcaccagcggccagagcgccggctggaagctgtggaagaatctggccgccaagagcgaaatcttcggcaagccggaaaagttctgcagcaatatcgaaaagagcgcctgggaaagcgccaccctgacctgcaagccgagcgccctgatcgataagctgaaggaatatagcgtgaatgatccgtatagcggcaagaccgtgaccggcggcatcatcaccatcaccgatagcaattggctgatgagcttcacctgcaatcgccagccgcatttcccggaacagccggatgatgtgctggtgctgtgggtgtatgccctgttcatggataaggaaggcaattatatcaagaagaccatgctggaatgcaccggcgatgaaatcctggccgaactgtgctatcatctgggcatcgaagatcagctggaaaatgtgcagaagaataccatcgtgcgcaccgccttcatgccgtatatcaccagcatgttcatgccgcgcgccaagggcgatcgcccgcgcgtggtgccggaaggctgcaagaatctgggcctggtgggccagttcgtggaaaccaataatgatgtggtgttcaccatggaaagcagcgtgcgcaccgcccgcatcgccgtgtataagctgctgaatctgaataagcaggtgccggatatcaatccgctgcagtatgatatccgccatctgctgaaggccgccaagaccctgaatgatgataagccgttcgtgggcgaaggcctgctgcgcaaggtgctgaagggcacctatttcgaacatgtgctgccggccggcgccgccgaagaagaagaacatgaaagcttcatcgccgaacatgtgaataagttccgcgaatgggtgaagggcatccgcggc

SEQ ID NO: 21 - DNA, 936 bp, Acidithiobacillus ferrooxidans
  source 1..936: mol_type = other DNA; note = carotenoid 1,2-hydratase (CruF) codon optimized for Acidithiobacillus ferrooxidans expression
atgatcagcccgccgctgctgcgctggggcctggccgccgccggcctgggcctggccttcctgggcgccctgctggccctggccgaacagagcgccggctgggccctgatcgccctgggcctgccgctgagcggcgtgttcgccctggccggcgatgccctgggcagcgccttcggcgccaccctgcgccgccgctggcagcgcctgctggccgaaaccccgccgtggctgggctggctggccctgagcgtggccctgaagatcccggtgccgctgtggccgcagggcttcgccctgctgggcctgctgagcaccggcgccctgttcgtggccggcctgagctatgccgcccagcgcgccggctggggccaggccgcccagctggccgccctggcctgcctggccggcctgggcgtggaactgctgggcagccgcaccggcgtgccgttcggccagtatagctatgccaccgccccggccccgaccgtgctgaccgtgccgctgatcgtgccgctgggctggttcgccttcaccctggccggcctgcagctggccggcggccgcccgtggctggccggcctgctgatcacctgctgggatgtgggcctggaaccgctgatgaccgcccagaattattggcgctggagcgatccgcatccgctgtgggccggcgccccggtgcagaatttcctgggctggtgggccgtgggcaccgccatcgcctggggcatgaagcgcctggccccgggcctgttcgatgccaaggaaaagcagaaggccgaaggcctgagcccgagcttcagcctggcctatttcaccgaagccttcttcctgccgggcggcctggtgctggtgggccgcctggccgaagccggcgtgaccctggccgtgatgggcctgggcgccctgctggcccgcggcgtgcgccgcgccagc

SEQ ID NO: 22 - DNA, 1218 bp, Acidithiobacillus ferrooxidans
  source 1..1218: mol_type = other DNA; note = carotenoid 1,2-hydratase (CrtC) codon optimized for Acidithiobacillus ferrooxidans expression
atgcgcgccgccgaaagcggcgccgatgcccgcgtgcgcccggtggatcgcgtggaaccggccgatgccccggccggcgatgccggcggcctgcgcgccgccgtgccgggcgatggcggcagcgccgtgcgcccgggcgatgcccgcctggatgtgctggtgccgccgggcctggtggatgaaccggccgccggcgccctgccgggcggcggccagcgcgccccgggcgccggccgcgccgatggcggcgatgtgcgcccggtgggcggccgcgatgccgatggcgccccgcgcttcgatcagccggtgccgccgggcggctatctgtggtggtatgtggatgccgtgagcgatgatggccgccatggcctgaccttcatcgccttcgtgggcagcgtgttcagcccgtattatgcctgggccggcggcccgaaggccgatcgcgccgatccggaaaatcattgcgccctgaatatcgccctgtatggcgatgccggcaagcgctggaccatgaccgaacgcggccgccgctggatgcgccgcagccgcgatgaattcgtgatcggcccgagccgcctgcattgggatggcgaaagcctgctggtggaattcgatgaagtgggcgtgccgatcccgcgccgcgtgaagggccgcgtgcgcgtgtggccgaaggccctgtgccgcttcgtgaccagcctggatagcggcggccgccatcgctggggcccgatcgccccgtgcagccgcatcgaagtggaactggatagcccgcgcgtgcgctggagcggccatgcctatctggatagcaatgaaggcgatgaaccgatcgatcgcccgttccgcgaatgggattggagccgcgccaccatggccgatagcagcaccgccgtgatctatgatgtgcgccagaagcgcgatggcgatcgcgtgatcgccgaacgcttcctgctggatggcagcaccgaaagcttcgaagccccgccgcgccagccgctgccgaccaccctgtggcgcatcggccgcaccatgcgcaccgaaccgggcgtgccggccctggtggaacagaccctggaagataccccgttctatgcccgcagcatggtgcgcagcggcctgctgggcgaagtggtgaccagcgtgcatgaaaccatgctgctgccgcgcgtgatcaccctgccggtgcgcctgatgctgccgtggcgcatgccgcgccgcgcc

SEQ ID NO: 23 - DNA, 1191 bp, Ferroplasma acidiphilum
  source 1..1191: mol_type = other DNA; note = linalool dehydratase codon optimized for Ferroplasma acidiphilum expression
atgaggtttacacttaaaacaacagcaatagtttccgcagcagcacttcttgcaggatttggacctcctcctagggcagcagaacttcctcctggaaggcttgcaacaacagaagattattttgcacagcaggcaaaacaggcagttacacctgatgttatggcacagcttgcatatatgaattatatagattttatatcccctttttattccaggggatgttcctttgaagcatgggaacttaaacatacacctcagagggttataaaatattccatagcattttatgcatatggacttgcatccgttgcacttatagatcctaaacttagggcacttgcaggacatgatcttgatatagcagtttccaaaatgaaatgtaaaagggtttggggagattgggaagaagatggatttggaacagatcctatagaaaaagaaaatataatgtataaaggacatcttaatcttatgtatggactttatcagcttgttacaggatccaggaggtatgaagcagaacatgcacatcttacaaggataatacatgatgaaatagcagcaaatccttttgcaggaatagtttgtgaacctgataattattttgttcagtgtaattccgttgcatatctttccctttgggtttatgataggcttcatggaacagattatagggcagcaacaagggcatggcttgattttatacagaaagatcttatagatcctgaaaggggagcattttatctttcctatcatcctgaatccggagcagttaaaccttggatatccgcatatacaacagcatggacacttgcaatggttcatggaatggatcctgcattttccgaaaggtattatcctaggtttaaacagacatttgttgaagtttatgatgaaggaaggaaagcaagggttagggaaacagcaggaacagatgatgcagatggaggagttggacttgcatccgcatttacacttcttcttgcaagggaaatgggagatcagcagctttttgatcagcttcttaatcatcttgaacctcctgcaaaaccttccatagtttccgcatcccttaggtatgaacatcctggatcccttctttttgatgaacttctttttcttgcaaaagttcatgcaggatttggagcacttcttaggatgcctcctcctgcagcaaaacttgcaggaaaa

SEQ ID NO: 24 - DNA, 1938 bp, Ferroplasma acidiphilum
  source 1..1938: mol_type = other DNA; note = oleate hydratase codon optimized for Ferroplasma acidiphilum expression
atgaatcctataacatccaaatttgataaagttcttaatgcatcctccgaatatggacatgttaatcatgaacctgattcctccaaagaacagcagaggaatacacctcagaaatccatgcctttttccgatcagataggaaattatcagaggaataaaggaatacctgttcagtcctatgataattccaaaatatatataataggatccggaatagcaggaatgtccgcagcatattattttataagggatggacatgttcctgcaaaaaatataacatttcttgaacagcttcatatagatggaggatcccttgatggagcaggaaatcctacagatggatatataataaggggaggaagggaaatggatatgacatatgaaaatctttgggatatgtttcaggatatacctgcacttgaaatgcctgcaccttattccgttcttgatgaatataggcttataaatgataatgattccaattattccaaagcaaggcttataaataataaaggagaaataaaagatttttccaaatttggacttaataaaatggatcagcttgcaataataaggcttcttcttaaaaataaagaagaacttgatgatcttacaatagaagattatttttccgaatcctttcttaaatccaatttttggacattttggaggacaatgtttgcatttgaaaattggcattcccttcttgaacttaaactttatatgcataggtttcttcatgcaatagatggacttaatgatctttcctcccttgtttttcctaaatataatcagtatgatacatttgttacacctcttaggaaatttcttcaggaaaaaggagttaatatacatcttaatacacttgttaaagatcttgatatacatataaatacagaaggaaaagttgttgaaggaataataacagaacaggatggaaaagaagttaaaatacctgttggaaaaaatgattatgttatagttacaacaggatccatgacagaagatacattttatggaaataataaaacagcacctataataggaatagataattccacatccggacagtccgcaggatggaaactttggaaaaatcttgcagcaaaatccgaaatatttggaaaacctgaaaaattttgttccaatatagaaaaatccgcatgggaatccgcaacacttacatgtaaaccttccgcacttatagataaacttaaagaatattccgttaatgatccttattccggaaaaacagttacaggaggaataataacaataacagattccaattggcttatgtcctttacatgtaataggcagcctcattttcctgaacagcctgatgatgttcttgttctttgggtttatgcactttttatggataaagaaggaaattatataaaaaaaacaatgcttgaatgtacaggagatgaaatacttgcagaactttgttatcatcttggaatagaagatcagcttgaaaatgttcagaaaaatacaatagttaggacagcatttatgccttatataacatccatgtttatgcctagggcaaaaggagataggcctagggttgttcctgaaggatgtaaaaatcttggacttgttggacagtttgttgaaacaaataatgatgttgtttttacaatggaatcctccgttaggacagcaaggatagcagtttataaacttcttaatcttaataaacaggttcctgatataaatcctcttcagtatgatataaggcatcttcttaaagcagcaaaaacacttaatgatgataaaccttttgttggagaaggacttcttaggaaagttcttaaaggaacatattttgaacatgttcttcctgcaggagcagcagaagaagaagaacatgaatcctttatagcagaacatgttaataaatttagggaatgggttaaaggaataagggga

SEQ ID NO: 25 - DNA, 936 bp, Ferroplasma acidiphilum
  source 1..936: mol_type = other DNA; note = carotenoid 1,2-hydratase (CruF) codon optimized for Ferroplasma acidiphilum expression
atgatatcccctcctcttcttaggtggggacttgcagcagcaggacttggacttgcatttcttggagcacttcttgcacttgcagaacagtccgcaggatgggcacttatagcacttggacttcctctttccggagtttttgcacttgcaggagatgcacttggatccgcatttggagcaacacttaggaggaggtggcagaggcttcttgcagaaacacctccttggcttggatggcttgcactttccgttgcacttaaaatacctgttcctctttggcctcagggatttgcacttcttggacttctttccacaggagcactttttgttgcaggactttcctatgcagcacagagggcaggatggggacaggcagcacagcttgcagcacttgcatgtcttgcaggacttggagttgaacttcttggatccaggacaggagttccttttggacagtattcctatgcaacagcacctgcacctacagttcttacagttcctcttatagttcctcttggatggtttgcatttacacttgcaggacttcagcttgcaggaggaaggccttggcttgcaggacttcttataacatgttgggatgttggacttgaacctcttatgacagcacagaattattggaggtggtccgatcctcatcctctttgggcaggagcacctgttcagaattttcttggatggtgggcagttggaacagcaatagcatggggaatgaaaaggcttgcacctggactttttgatgcaaaagaaaaacagaaagcagaaggactttccccttccttttcccttgcatattttacagaagcattttttcttcctggaggacttgttcttgttggaaggcttgcagaagcaggagttacacttgcagttatgggacttggagcacttcttgcaaggggagttaggagggcatcc

SEQ ID NO: 26 - DNA, 1218 bp, Ferroplasma acidiphilum
  source 1..1218: mol_type = other DNA; note = carotenoid 1,2-hydratase (CrtC) codon optimized for Ferroplasma acidiphilum expression
atgagggcagcagaatccggagcagatgcaagggttaggcctgttgatagggttgaacctgcagatgcacctgcaggagatgcaggaggacttagggcagcagttcctggagatggaggatccgcagttaggcctggagatgcaaggcttgatgttcttgttcctcctggacttgttgatgaacctgcagcaggagcacttcctggaggaggacagagggcacctggagcaggaagggcagatggaggagatgttaggcctgttggaggaagggatgcagatggagcacctaggtttgatcagcctgttcctcctggaggatatctttggtggtatgttgatgcagtttccgatgatggaaggcatggacttacatttatagcatttgttggatccgttttttccccttattatgcatgggcaggaggacctaaagcagatagggcagatcctgaaaatcattgtgcacttaatatagcactttatggagatgcaggaaaaaggtggacaatgacagaaaggggaaggaggtggatgaggaggtccagggatgaatttgttataggaccttccaggcttcattgggatggagaatcccttcttgttgaatttgatgaagttggagttcctatacctaggagggttaaaggaagggttagggtttggcctaaagcactttgtaggtttgttacatcccttgattccggaggaaggcataggtggggacctatagcaccttgttccaggatagaagttgaacttgattcccctagggttaggtggtccggacatgcatatcttgattccaatgaaggagatgaacctatagataggccttttagggaatgggattggtccagggcaacaatggcagattcctccacagcagttatatatgatgttaggcagaaaagggatggagatagggttatagcagaaaggtttcttcttgatggatccacagaatcctttgaagcacctcctaggcagcctcttcctacaacactttggaggataggaaggacaatgaggacagaacctggagttcctgcacttgttgaacagacacttgaagatacacctttttatgcaaggtccatggttaggtccggacttcttggagaagttgttacatccgttcatgaaacaatgcttcttcctagggttataacacttcctgttaggcttatgcttccttggaggatgcctaggagggca

SEQ ID NO: 27 - DNA, 1191 bp, Acidithiobacillus thiooxidans
  source 1..1191: mol_type = other DNA; note = linalool dehydratase codon optimized for Acidithiobacillus thiooxidans expression
atgcgtttcaccctgaaaaccaccgccattgtgagcgccgccgccctgctggccggtttcggtccgccgccgcgtgccgccgaactgccgccgggtcgtctggccaccaccgaagattacttcgcccagcaggccaaacaggccgtgaccccggatgtgatggcccagctggcctacatgaattacattgatttcattagcccgttctacagccgtggttgcagcttcgaagcctgggaactgaaacacaccccgcagcgtgtgattaaatacagcattgccttctacgcctacggtctggccagcgtggccctgattgatccgaaactgcgtgccctggccggtcacgatctggatattgccgtgagcaaaatgaaatgcaaacgtgtgtggggtgattgggaagaagatggtttcggtaccgatccgattgaaaaagaaaatattatgtacaaaggtcacctgaatctgatgtacggtctgtaccagctggtgaccggtagccgtcgttacgaagccgaacacgcccacctgacccgtattattcacgatgaaattgccgccaatccgttcgccggtattgtgtgcgaaccggataattacttcgtgcagtgcaatagcgtggcctacctgagcctgtgggtgtacgatcgtctgcacggtaccgattaccgtgccgccacccgtgcctggctggatttcattcagaaagatctgattgatccggaacgtggtgccttctacctgagctaccacccggaaagcggtgccgtgaaaccgtggattagcgcctacaccaccgcctggaccctggccatggtgcacggtatggatccggccttcagcgaacgttactacccgcgtttcaaacagaccttcgtggaagtgtacgatgaaggtcgtaaagcccgtgtgcgtgaaaccgccggtaccgatgatgccgatggtggtgtgggtctggccagcgccttcaccctgctgctggcccgtgaaatgggtgatcagcagctgttcgatcagctgctgaatcacctggaaccgccggccaaaccgagcattgtgagcgccagcctgcgttacgaacacccgggtagcctgctgttcgatgaactgctgttcctggccaaagtgcacgccggtttcggtgccctgctgcgtatgccgccgccggccgccaaactggccggtaaa

SEQ ID NO: 28 - DNA, 1938 bp, Acidithiobacillus thiooxidans
  source 1..1938: mol_type = other DNA; note = oleate hydratase codon optimized for Acidithiobacillus thiooxidans expression
atgaatccgattaccagcaaattcgataaagtgctgaatgccagcagcgaatacggtcacgtgaatcacgaaccggatagcagcaaagaacagcagcgtaataccccgcagaaaagcatgccgttcagcgatcagattggtaattaccagcgtaataaaggtattccggtgcagagctacgataatagcaaaatttacattattggtagcggtattgccggtatgagcgccgcctactacttcattcgtgatggtcacgtgccggccaaaaatattaccttcctggaacagctgcacattgatggtggtagcctggatggtgccggtaatccgaccgatggttacattattcgtggtggtcgtgaaatggatatgacctacgaaaatctgtgggatatgttccaggatattccggccctggaaatgccggccccgtacagcgtgctggatgaataccgtctgattaatgataatgatagcaattacagcaaagcccgtctgattaataataaaggtgaaattaaagatttcagcaaattcggtctgaataaaatggatcagctggccattattcgtctgctgctgaaaaataaagaagaactggatgatctgaccattgaagattacttcagcgaaagcttcctgaaaagcaatttctggaccttctggcgtaccatgttcgccttcgaaaattggcacagcctgctggaactgaaactgtacatgcaccgtttcctgcacgccattgatggtctgaatgatctgagcagcctggtgttcccgaaatacaatcagtacgataccttcgtgaccccgctgcgtaaattcctgcaggaaaaaggtgtgaatattcacctgaataccctggtgaaagatctggatattcacattaataccgaaggtaaagtggtggaaggtattattaccgaacaggatggtaaagaagtgaaaattccggtgggtaaaaatgattacgtgattgtgaccaccggtagcatgaccgaagataccttctacggtaataataaaaccgccccgattattggtattgataatagcaccagcggtcagagcgccggttggaaactgtggaaaaatctggccgccaaaagcgaaattttcggtaaaccggaaaaattctgcagcaatattgaaaaaagcgcctgggaaagcgccaccctgacctgcaaaccgagcgccctgattgataaactgaaagaatacagcgtgaatgatccgtacagcggtaaaaccgtgaccggtggtattattaccattaccgatagcaattggctgatgagcttcacctgcaatcgtcagccgcacttcccggaacagccggatgatgtgctggtgctgtgggtgtacgccctgttcatggataaagaaggtaattacattaaaaaaaccatgctggaatgcaccggtgatgaaattctggccgaactgtgctaccacctgggtattgaagatcagctggaaaatgtgcagaaaaataccattgtgcgtaccgccttcatgccgtacattaccagcatgttcatgccgcgtgccaaaggtgatcgtccgcgtgtggtgccggaaggttgcaaaaatctgggtctggtgggtcagttcgtggaaaccaataatgatgtggtgttcaccatggaaagcagcgtgcgtaccgcccgtattgccgtgtacaaactgctgaatctgaataaacaggtgccggatattaatccgctgcagtacgatattcgtcacctgctgaaagccgccaaaaccctgaatgatgataaaccgttcgtgggtgaaggtctgctgcgtaaagtgctgaaaggtacctacttcgaacacgtgctgccggccggtgccgccgaagaagaagaacacgaaagcttcattgccgaacacgtgaataaattccgtgaatgggtgaaaggtattcgtggt

SEQ ID NO: 29 - DNA, 936 bp, Acidithiobacillus thiooxidans
  source 1..936: mol_type = other DNA; note = carotenoid 1,2-hydratase (CruF) codon optimized for Acidithiobacillus thiooxidans expression
atgattagcccgccgctgctgcgttggggtctggccgccgccggtctgggtctggccttcctgggtgccctgctggccctggccgaacagagcgccggttgggccctgattgccctgggtctgccgctgagcggtgtgttcgccctggccggtgatgccctgggtagcgccttcggtgccaccctgcgtcgtcgttggcagcgtctgctggccgaaaccccgccgtggctgggttggctggccctgagcgtggccctgaaaattccggtgccgctgtggccgcagggtttcgccctgctgggtctgctgagcaccggtgccctgttcgtggccggtctgagctacgccgcccagcgtgccggttggggtcaggccgcccagctggccgccctggcctgcctggccggtctgggtgtggaactgctgggtagccgtaccggtgtgccgttcggtcagtacagctacgccaccgccccggccccgaccgtgctgaccgtgccgctgattgtgccgctgggttggttcgccttcaccctggccggtctgcagctggccggtggtcgtccgtggctggccggtctgctgattacctgctgggatgtgggtctggaaccgctgatgaccgcccagaattactggcgttggagcgatccgcacccgctgtgggccggtgccccggtgcagaatttcctgggttggtgggccgtgggtaccgccattgcctggggtatgaaacgtctggccccgggtctgttcgatgccaaagaaaaacagaaagccgaaggtctgagcccgagcttcagcctggcctacttcaccgaagccttcttcctgccgggtggtctggtgctggtgggtcgtctggccgaagccggtgtgaccctggccgtgatgggtctgggtgccctgctggcccgtggtgtgcgtcgtgccagc

SEQ ID NO: 30 - DNA, 1218 bp, Acidithiobacillus thiooxidans
  source 1..1218: mol_type = other DNA; note = carotenoid 1,2-hydratase (CrtC) codon optimized for Acidithiobacillus thiooxidans expression
atgcgtgccgccgaaagcggtgccgatgcccgtgtgcgtccggtggatcgtgtggaaccggccgatgccccggccggtgatgccggtggtctgcgtgccgccgtgccgggtgatggtggtagcgccgtgcgtccgggtgatgcccgtctggatgtgctggtgccgccgggtctggtggatgaaccggccgccggtgccctgccgggtggtggtcagcgtgccccgggtgccggtcgtgccgatggtggtgatgtgcgtccggtgggtggtcgtgatgccgatggtgccccgcgtttcgatcagccggtgccgccgggtggttacctgtggtggtacgtggatgccgtgagcgatgatggtcgtcacggtctgaccttcattgccttcgtgggtagcgtgttcagcccgtactacgcctgggccggtggtccgaaagccgatcgtgccgatccggaaaatcactgcgccctgaatattgccctgtacggtgatgccggtaaacgttggaccatgaccgaacgtggtcgtcgttggatgcgtcgtagccgtgatgaattcgtgattggtccgagccgtctgcactgggatggtgaaagcctgctggtggaattcgatgaagtgggtgtgccgattccgcgtcgtgtgaaaggtcgtgtgcgtgtgtggccgaaagccctgtgccgtttcgtgaccagcctggatagcggtggtcgtcaccgttggggtccgattgccccgtgcagccgtattgaagtggaactggatagcccgcgtgtgcgttggagcggtcacgcctacctggatagcaatgaaggtgatgaaccgattgatcgtccgttccgtgaatgggattggagccgtgccaccatggccgatagcagcaccgccgtgatttacgatgtgcgtcagaaacgtgatggtgatcgtgtgattgccgaacgtttcctgctggatggtagcaccgaaagcttcgaagccccgccgcgtcagccgctgccgaccaccctgtggcgtattggtcgtaccatgcgtaccgaaccgggtgtgccggccctggtggaacagaccctggaagataccccgttctacgcccgtagcatggtgcgtagcggtctgctgggtgaagtggtgaccagcgtgcacgaaaccatgctgctgccgcgtgtgattaccctgccggtgcgtctgatgctgccgtggcgtatgccgcgtcgtgcc

(19) United States
(12) Patent Application Publication
(10) Pub. No.: US 2024/0035050 A1
(43) Pub. Date: Feb. 1, 2024

(54) PRODUCT

(71) Applicant: CEMVITA FACTORY, INC., HOUSTON, TX (US)

(72) Inventors: Tahereh KARIMI, Houston, TX (US); Nymul KHAN, Houston, TX (US); Davi Ludvig GONÇALVES, HOUSTON, TX (US); Kevin ROBERG-PEREZ, HOUSTON, TX (US)

(21) Appl. No.: 18/325,786

(22) Filed: May 30, 2023

Related U.S. Application Data
(60) Provisional application No. 63/365,503, filed on May 31, 2022.

(51) Int. Cl. C12P 5/02; C12N 15/52; C12N 9/02; C12N 9/10; C12P 7/06

(52) U.S. Cl. CPC C12P 5/026; C12N 15/52; C12N 9/0095; C12Y 205/01006; C12N 9/1085; C12P 7/06

(57) ABSTRACT

The present application discloses a microorganism engineered to express one or more enzymes which enhances the microorganism's ability to produce C₂₋₄ alkene, and/or ethylene from C₂₋₄ alkanol or thiol, optionally ethanol. The process of producing C₂₋₄ alkanol and/or ethylene by the engineered microorganism is also discussed.

This application claims priority to U.S. Provisional Patent Application No. 63/365,503, filed May 31, 2022.

### FIELD

The present application provides microorganisms useful in the production of alkene, such as ethylene. Alkene production methods employing such microorganisms are also provided.

### BACKGROUND

The limited supply of petroleum and its harmful effects on the environment have prompted developments in renewable sources of fuels and chemicals. Ethylene is the most widely produced organic compound in the world, useful in a broad spectrum of industries including plastics, solvents, and textiles. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. Producing ethylene through renewable methods would accordingly help to meet the huge demand from the energy and chemical industries, while also helping to protect the environment.

Since ethylene is a potentially renewable feedstock, there has been a great deal of interest in developing technologies to produce ethylene from renewable sources.

One well established technique is a two-step process in which bioethanol is produced using microorganisms such as Saccharomyces cerevisiae which efficiently produce ethanol from sugars in significant amounts. The obtained bioethanol is then converted to ethylene via a chemical dehydration process.

However, the dehydration step is energy intensive with a relatively high carbon dioxide footprint. Additionally, the performance of i) a microbial step and ii) an energy intensive chemical reaction step makes the process capital intensive.

One approach to improve the efficiency of the bioproduction of ethylene has been to engineer microorganisms to express ethylene forming enzyme (EFE). While somewhat successful, one major drawback of systems utilizing such organisms is that the EFE enzyme functions by catalyzing the conversion of alpha-ketoglutarate to ethylene with oxygen being required to drive this reaction forwards. The requirement for the presence of oxygen is problematic for two reasons. Firstly, there are significant safety issues with scaling up systems comprising ethylene (formed by the EFE enzyme) and oxygen (which must be supplied to the EFE system) as both are explosive gases. Secondly, the requirement to provide the system with oxygen means that the use of anaerobic organisms as the chassis for expressing EFE is not possible, thus reducing the options available to the skilled person when designing novel recombinant EFE-expressing organisms.

There remains a need for improvements in microbial bioethylene systems and processes, in order to produce ethylene and other alkenes at a commercial scale. There also remains a need to produce hydrocarbons through more efficient renewable technologies.

There additionally remains a need for improved methods to produce ethylene from a renewable feedstock for industrial and commercial applications. One, some or all of these needs are addressed by the present application.

### SUMMARY

Thus, according to a first aspect of the present application there is provided a microorganism engineered to express one or more enzymes which enhances the microorganism's ability to produce C₂₋₄ alkene such as ethylene from a C₂₋₄ alkanol or thiol such as ethanol.

As used herein, reference to a microorganism's ability to produce C₂₋₄ alkene such as ethylene from C₂₋₄ alkanol or thiol being enhanced is relative to the wild type strain. Accordingly, if a microorganism is engineered, but this does not result in its ability to produce C₂₋₄ alkene such as ethylene from C₂₋₄ alkanol or thiol being enhanced as compared to the unengineered strain, then it would not exhibit this feature. For example, if a wild type microorganism expresses one or more enzymes which enable it to produce C₂₋₄ alkene such as ethylene from C₂₋₄ alkanol or thiol and the microorganism is then engineered to i) remove or inactivate (e.g. by ‘knocking out’, deleting or mutating the respective gene/s) its ability to express those one or more enzymes and, subsequently, to ii) reactivate or revive (e.g. by ‘knocking in’, adding or mutating the respective gene/s) its ability to express those one or more enzymes at the same level as the unengineered, wild type organism, then it would not be considered to have enhanced ability to convert C₂₋₄ alkanol or thiol to C₂₋₄ alkene such as ethylene.

As used herein, C₂₋₄ alkanol or thiol encompasses all aliphatic alkanols and thiols comprising a 2 to 4 carbon atom backbone. Examples of C₂₋₄ alkanols or thiols include ethanol, propanol, butanol, ethane thiol, propane thiol and butane thiol. Compounds comprising multiple —OH and/or —SH groups are also encompassed by this term, such as diols, triols, dithiols, trithiols. In preferred embodiments, the C₂₋₄ alkanol or thiol is ethanol.

The inventors have identified that engineering an organism to express an enzyme, for example, one from the S-Adenosyl methionine (SAM) pathway or a dehydratase, which enhances the microorganism's ability to produce C₂₋₄ alkene such as ethylene from C₂₋₄ alkanol or thiol such as ethanol is advantageous for several reasons. Firstly, it provides a substantial improvement over conventional systems in which a chemical, energy intensive, dehydration step is employed to convert a C₂₋₄ alkanol or thiol such as bioethanol to C₂₋₄ alkene such as ethylene.

Secondly, through the use of such enzymes, the enzymatic route via which the production of C₂₋₄ alkenes such as ethylene may be achieved is decoupled from the Krebs cycle. Thus, the need to provide systems which express EFE and thus need to be supplied with oxygen can be avoided which makes scale up less hazardous. Further, systems employing the organism of the invention may be anaerobic which is a major benefit in the production of volatile compounds such as ethylene.

In embodiments of the invention, the enzyme expressed by the engineered microorganism may be part of the S-Adenosyl methionine pathway. In certain embodiments, the microorganism of the invention may be engineered to express methylthio-alkane reductase, MarBHDK and/or Cys, Met/SAM synthase.

In certain embodiments of the invention, the enzyme of the S-Adenosyl methionine pathway which the microorganism of the invention may be engineered to express may utilize 2-(methylthio) ethanol as substrate and convert this substrate into ethylene and methylthionine. Additionally or alternatively, the enzyme of the S-Adenosyl methionine pathway which the microorganism of the invention may be engineered to express may utilize methanethiol and ethanol as substrates and convert these substrates into ethylene and methylthionine. Such reactions may occur via the following reaction:

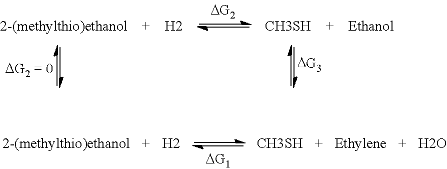

In embodiments of the invention, the enzyme expressed by the engineered microorganism may be a dehydratase enzyme. As those skilled in the art will recognize, dehydratase enzymes are capable of catalysing the dehydration of carbon oxygen linkages. It will also be recognized that dehydratase enzymes may also have the ability to catalyse the reverse reaction, i.e. the hydration of carbon oxygen linkages and thus, in embodiments, the dehydratase enzyme may have hydratase activity. In such embodiments, the enzyme may have preferential dehydratase vs hydratase activity.

In embodiments of the invention, the dehydratase enzyme may be an unsaturated fatty acid dehydratase enzyme, such as oleate dehydratase. Additionally or alternatively, the dehydratase may be an alkenol dehydratase, e.g. linalool dehydratase. In certain embodiments, the dehydratase enzyme may be a carotenoid dehydratase, for example one utilizing a cyclic substrate (e.g. cruF, optionally obtained from Deinococcus radiodurans) or one utilizing an acyclic substrate (e.g. crtC optionally obtained from Rubrivivax gelatinosus).

To the inventors' knowledge, the engineering of microorganisms to express dehydratase enzymes useful in the conversion of ethanol to ethylene has not previously been disclosed in an enabled manner. While there have been references to engineering dehydratase activity into microorganisms in the literature, the actual preparation of microorganisms having this functionality has not been meaningfully exemplified until now. For example, in Example 55 of WO2009/111513, it is noted that alcohol dehydratase converts ethanol to ethylene. However, the engineering of a microorganism to express such an enzyme is not disclosed, even at a high level. Similarly, in Example 10 of WO2011/076691, the transformation of unspecified ‘ethanol producing E. coli bacteria’ with plasmids encoding unspecified enzymes having activity in converting ethanol to ethylene is proposed, but without any clear guidance as to how this should be completed.

The microorganism of the present application may be of any type, including bacteria, archaea, fungi (e.g. yeasts), protists, or the like. Especially preferred microorganisms are bacteria and fungi.

For the avoidance of doubt, where reference is made herein to the engineering of a microorganism to express an enzyme, this encompasses the engineering of a microorganism to express a non-native enzyme to the microorganism (i.e. which is not expressed by the wild-type microorganism) and/or the overexpression of an enzyme which is native to the microorganism (i.e. which is expressed by the wild type organism, but in lower amounts than in the engineered microorganism).

Those skilled in the art will be familiar with techniques for engineering microorganisms. In preferred embodiments, microorganism may be genetically manipulated, for example the genome of the microorganism may be modified, and/or the microorganism may be transformed using a plasmid.

As explained herein, the engineered microorganisms of the present application are particularly useful in the production of C₂₋₄ alkene such as ethylene. Thus, according to a further aspect of the present application, there is provided a process for producing C₂₋₄ alkene such as ethylene comprising i) contacting a C₂₋₄ alkanol or thiol such as ethanol with an enzyme capable of catalysing the conversion of the C₂₋₄ alkanol or thiol to C₂₋₄ alkene expressed by a microorganism, and ii) obtaining C₂₋₄ alkene.

In such embodiments, the microorganism may be a microorganism of the invention, i.e. one as discussed herein. For example, the microorganism may be engineered to express an enzyme which enhances the microorganism's ability to produce C₂₋₄ alkene such as ethylene from C₂₋₄ alkanol or thiol such as ethanol (e.g. one belonging to the S-Adenosyl methionine synthesis pathway or a dehydratase enzyme). In alternative embodiments, the microorganism may be native, i.e. unengineered, having the innate ability to catalyse the conversion of C₂₋₄ alkanol or thiol to C₂₋₄ alkene.

According to a further aspect of the present application, there is provided a composition comprising a microorganism of the present application and a carrier. In such embodiments, the composition may be in the form of a live culture, a frozen culture, an inoculum or a lyophilised composition.

### BRIEF DESCRIPTION OF THE DRAWINGS

FIG. 1 shows Colony PCR screening using plasmid DNA from 21 different S. cerevisiae candidates and the results. The left hand side of the FIGURE, was estimated using.

### DETAILED DESCRIPTION

Herein incorporated by reference is the sequence listing filed with the USPTO as P12965WO—Sequence listing.xml which was created on May 28, 2023, and the size is 73,807 bytes.

Unless otherwise noted, all instances of the words “a,” “an,” or “the” can refer to one or more than one of the word that they modify.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by various methods, known to those skilled in the art. In an embodiment, sequence identity is determined by comparing the whole length of the sequences as identified herein.

As those skilled in the art will recognize, the S-Adenosyl-L-methionine (SAM) pathway is the biosynthetic pathway via methionine is converted into S-Adenosyl-L-methionine which operates in microorganisms such as Rhodopseudomonas palustris and Rhodospirillum rubrum. Further information on this pathway and the enzymes comprised therein can be found in North et al., Molecular Microbiology, 2020, 113: 923-937 and North et al., Science, 1094-1098 (2020).

Exemplary methods to determine identity are designed to give the largest match between the sequences tested. Methods to determine identity and similarity are codified in publicly available computer programs. Exemplary computer program methods to determine identity and similarity between two sequences include e.g., the BestFit, BLASTP (Protein Basic Local Alignment Search Tool), BLASTN (Nucleotide Basic Local Alignment Search Tool), and FASTA (Altschul, S. F. et al., J. Mol. Biol. 215:403-410 (1990), publicly available from NCBI and other sources (BLAST® Manual, Altschul, S., et al., NCBI NLM NIH Bethesda, Md. 20894). A most exemplary algorithm used is EMBOSS (European Molecular Biology Open Software Suite). Exemplary parameters for amino acid sequences comparison using EMBOSS are gap open 10.0, gap extend 0.5, BLOSUM matrix. Exemplary parameters for nucleic acid sequences comparison using EMBOSS are gap open 10.0, gap extend 0.5, DNA full matrix (DNA identity matrix). In embodiments, it is possible to compare the DNA/protein sequences among different species to determine the homology of sequences using online data such as Gene bank, KEGG, BLAST and Ensemble.

### Microorganisms

The microorganism of the present application may be of any type, including bacteria, archaea, fungi (e.g. yeasts), protists (e.g., protozoa, algae) or the like, provided that they are capable of catalysing the conversion of C₂₋₄ alkanol or thiol such as ethanol to C₂₋₄ alkene such as ethylene.

In embodiments, the microorganism is anaerobic. For example, the microorganism may be facultitatively anaerobic and/or obligately anaerobic. Advantageously, the use of anaerobic organisms enables the production of C₂₋₄ alkene such as ethylene in the absence of oxygen, this making such reactions safer.

Additionally or alternatively, the microorganism may be chemotrophic, for example chemoorganotrophic or chemolithotrophic. In some embodiments, the microorganism may be an iron oxidiser, a sulfur oxidiser and/or a denitrifyer.

Examples of bacteria for use in the present application include those of the Rhodobacteriaceae family, including from the genera Paracoccus (e.g. Paracoccus denitrificans), Rhodobacter (e.g. Rhodobacter capsulatus), Rhodospirillum (e.g. Rhodospirillum rubrum), and Rhodopseudomonas (e.g. Rhodopseudomonas palustris), the Pseudomonaceae family, including from the genera Azotobacter (e.g. Azotobacter vinelandii) and Pseudomonas (e.g. Pseudomonas putida), the Blastochloridaceae family, including from the genus Blastochloris (e.g. Blastochloris viridis), the Enterobacteriaceae family, including from the Escherichia genus (e.g. Escherichia coli) the Endomicrobaceae family, including from the genus Endomicrobium (e.g. Endomicrobium proavitum), the Acidithiobacillaceae family, including from the genus Acidithiobacillus (e.g. Acidithiobacillus ferrooxidans or Acidithiobacillus thiooxidans), the Ferroplasmaceae family, including from the genus Ferroplasma (e.g. Ferroplasma acidiphilum), the Nitrospiraceae family, including from the genus Nitrospira (e.g. Nitrospira japonica), the Nitrobacteraceae family, including from the genus Nitrobacter (e.g. Nitrobacter winogradskyi).

In embodiments of the invention, the microorganism of the present application may be a yeast. In specific embodiments, the yeast may be a C₂₋₄ alkanol or thiol producing yeast, for example one belonging to the genus Saccharomyces (e.g. Saccharomyces cerevisiae).

The microorganism may have the ability to produce C₂₋₄ alkanol or thiol such as ethanol. In some embodiments, the microorganism's ability to produce C₂₋₄ alkanol or thiol may be native, i.e. the microorganism innately produces the C₂₋₄ alkanol or thiol. In alternative embodiments, the microorganism may be engineered to produce C₂₋₄ alkanol or thiol. For example, the microorganism may be engineered to introduce a non-native gene expressing an C₂₋₄ alkanol or thiol producing enzyme. Alternatively, the microorganism may be engineered to overexpress an C₂₋₄ alkanol or thiol producing enzyme expressed by a gene native to the microorganism.

### Engineering

In embodiments of the invention, the microorganism may be engineered in any way or using any technique which can enhance its ability to catalyse the conversion of C₂₋₄ alkanol or thiol such as ethanol to C₂₋₄ alkene such as ethylene.

For example, the microorganism may be engineered through the direct modification of its genome. Additionally, or alternatively, the microorganism may be engineered via transformation using a plasmid. In such embodiments, engineering of the bacteria may involve the addition, introduction, insertion or knocking in of one or more genes expressing enzymes capable of catalysing the conversion of C₂₋₄ alkanol or thiol to C₂₋₄ alkene such as ethylene. Said one or more genes may be present in the native strain, or may be a gene not present in the native strain. In some embodiments, the gene may have 80% or higher, 85% or higher, 90% or higher, 95% or higher, 97% or higher, 98% or higher, 99% or higher, 99.5% or higher, 99.7% or higher, 99.8% or higher or 99.9% or higher sequence identity to any of SEQ ID NOs. 1 to 30.

In preferred embodiments of the present application, the microorganism is engineered to express an enzyme from the S-Adenosyl-L-methionine (SAM) pathway. For example, the microorganism may be engineered to express methylthio-alkane reductase, MarBHDK and/or Cys, Met/SAM synthase. Additionally or alternatively, the microorganism may be engineered to overexpress methylthio-alkane reductase, MarBHDK and/or Cys, Met/SAM synthase.

Additionally or alternatively, in embodiments of the invention, the microorganism is engineered to express a dehydratase enzyme. The dehydratase enzyme may also have hydratase functionality. In embodiments, the dehydratase enzyme has preferential dehydratase vs hydratase activity.

In certain embodiments, the microorganism may be engineered to express a dehydratase, such as an unsaturated fatty acid dehydratase (e.g. oleate dehydratase), an alkenol dehydratase (e.g. linalool dehydratase) and/or a carotenoid dehydratase (e.g. one utilizing a cyclic substrate (e.g. CruF) or an acyclic substrate (e.g. CrtC). Additionally or alternatively, the microorganism may be engineered to overexpress a dehydratase, such as an unsaturated fatty acid dehydratase (e.g. oleate dehydratase), an alkenol dehydratase (e.g. linalool dehydratase) and/or a carotenoid dehydratase (e.g. one utilizing a cyclic substrate (e.g. CruF) or an acyclic substrate (e.g. CrtC).

Such genes may be derived from the same strain or species as the microorganism of the invention or may be derived from alternative strains or species.

In embodiments of the invention, the microorganism does not express EFE. In certain embodiments, the microorganism is not engineered to express EFE. In some embodiments, the wild type microorganism does not express EFE.

Additionally or alternatively, the microorganism does not express an enzyme which utilizes oxygen to produce C₂₋₄ alkene such as ethylene. In certain embodiments, the microorganism is not engineered to express an enzyme which utilizes oxygen to produce C₂₋₄ alkene such as ethylene. In some embodiments, the wild type organism does not express an enzyme which utilizes oxygen to produce C₂₋₄ alkene such as ethylene.

In embodiments, the microorganism does not express an enzyme which produces oxygen and C₂₋₄ alkene such as ethylene. In certain embodiments, the microorganism is not engineered to express an enzyme which produces oxygen and C₂₋₄ alkene such as ethylene. In some embodiments, the wild type organism does not express an enzyme which produces oxygen and C₂₋₄ alkene such as ethylene.

In certain embodiments, the enzyme which enhances the microorganism's ability to produce C₂₋₄ alkene does not utilise and/or produce oxygen.

As discussed above, in embodiments of the present application, plasmids may be utilized to engineer the microorganisms of the present application via transformation. Any plasmid capable of transforming the microorganism of interest may be employed. Exemplary plasmids which may be employed include p416TEF1 and p416TPI1.

In such embodiments, the plasmid preferably comprises a promoter (e.g. trc, psbA, LacUV5, TEF1, TPI), a gene to be transformed into the microorganism of interest (e.g. one expressing the an enzyme from the S-Adenosyl-L-methionine (SAM) pathway, e.g. methylthio-alkane reductase, MarBHDK and/or Cys, Met/SAM synthase, or a dehydratase as discussed herein), and a terminator (e.g. T7, rrnB).

### Methods of Application

As a result of their being engineered to enhance their ability to catalyse the conversion of C₂₋₄ alkanol or thiol to C₂₋₄ alkene, the microorganisms of the present application are suitable for use in the production of C₂₋₄ alkene such as ethylene. Thus, according to a further aspect of the present application, there is provided a process for producing C₂₋₄ alkene such as ethylene comprising i) contacting C₂₋₄ alkanol or thiol such as ethanol with an enzyme capable of catalysing the conversion of C₂₋₄ alkanol or thiol to C₂₋₄ alkene such as ethylene expressed by a microorganism, and ii) obtaining C₂₋₄ alkene such as ethylene.

In such embodiments, the microorganism may be a microorganism of the invention, i.e. one which has been engineered to enhance its ability to catalyse the conversion of C₂₋₄ alkanol or thiol such as ethanol to C₂₋₄ alkene such as ethylene. In alternative embodiments, the microorganism may be native, i.e. unengineered, having the innate ability to express an enzyme which catalyses the conversion of C₂₋₄ alkanol or thiol to C₂₋₄ alkene.

In embodiments of the invention, the process may comprise the step of collecting an enzyme capable of catalysing the conversion of C₂₋₄ alkanol or thiol such as ethanol to C₂₋₄ alkene such as ethylene from a microorganism. This step may be performed prior to and/or simultaneous with the step of contacting C₂₋₄ alkanol or thiol with an enzyme. In embodiments of the invention, the processes of the invention may be ‘cell free’, i.e. the microorganism from which the enzyme capable of catalysing the conversion of C₂₋₄ alkanol or thiol to C₂₋₄ alkene such as ethylene is not present when the enzyme is contacted with the C₂₋₄ alkanol or thiol. Additionally or alternatively, the invention may comprise the step of contacting the microorganism and the enzyme with the C₂₋₄ alkanol or thiol.

The C₂₋₄ alkanol or thiol such as ethanol may be provided via any means. In embodiments of the invention, the C₂₋₄ alkanol or thiol may be produced by the microorganism. In such embodiments, the microorganism may be a yeast which is capable of producing C₂₋₄ alkanol or thiol such as ethanol. Additionally or alternatively, the microorganism may be engineered to enhance its ability to produce C₂₋₄ alkanol or thiol. In certain embodiments, C₂₋₄ alkanol or thiol may be produced using a different microorganism to that employed in step i) of the process described above. In some embodiments, the C₂₋₄ alkanol or thiol may be abiotically produced.

The process of the invention may be carried out in a range of settings, including those used conventionally in C₂₋₄ alkene (e.g. ethylene) production processes.

In embodiments of the invention, step i) of the process may be operated in a reactor, for example, a tank, chamber or other type of container. In such embodiments, the enzyme and/or the microorganism expressing the enzyme may be fed into the reactor. In ‘cell free’ embodiments, the enzyme but not the microorganism may be fed into the reactor.

In certain embodiments, C₂₋₄ alkanol or thiol such as ethanol may be fed into the reactor.

In embodiments of the invention, oxygen is not fed into reactor.

In specific embodiments, a nutrition source may be fed into the reactor, for example a carbohydrate source (e.g. a monosaccharide, a disaccharide, an oligosaccharide and/or a polysaccharide), a nitrogen source (e.g. an amino acid source and/or a protein source) and/or a phosphorous source (e.g. phosphate).

In embodiments of the invention, the process may be conducted under controlled temperature. For example, step i) may be conducted (partially or completely) at a temperature of ≥10° C., ≥15° C. or ≥20° C. Additionally, or alternatively, step i) may be conducted (partially or completely) at a temperature of ≤80° C., ≤70° C., ≤60° C., or ≤50° C. In certain embodiments, step i) may be conducted (partially or completely) at a temperature of about 20° C. to about 45° C. or from about 25° C. to about 40° C. In alternative embodiments (for example, in a cell free process) step i) may be conducted (partially or completely) at a temperature of about 35° C. to about 60° C. or from about 40° C. to about 55° C.

In embodiments of the invention, step i) of the process may be conducted in a reaction medium. In such embodiments, the reaction medium may have a volume of at least 5 mL, at least 10 mL, at least 50 mL, at least 100 mL, at least 500 mL, at least 1000 mL, at least 5 L, at least 10 L, at least 50 L, at least 100 L, at least 500 L or at least 1000 L.

The process of the invention may be worked in low oxygen environments, thus making the production of C₂₋₄ alkene such as ethylene safer. In embodiments of the invention, step i) of the process of the invention may be conducted under anaerobic conditions. For example, the process of the invention may be conducted under an atmosphere comprising oxygen at a level of about 10% or less, about 8% or less, about 6% or less, about 5% or less, about 4% or less, about 3% or less, about 2% or less or about 1% or less. Anaerobic conditions may be achieved by purging the reactor in which that step is performed with an inert gas, e.g. nitrogen or argon, and/or exposing the reactor to a vacuum. In embodiments of the invention, the environment in which the process of the invention is worked is not aerated.

### Compositions

According to a further aspect of the present application, there is provided a composition comprising a microorganism engineered to enhance its ability to catalyse the conversion of C₂₋₄ alkanol or thiol such as ethanol to C₂₋₄ alkene such as ethylene and a carrier.

In such embodiments, the composition may be in the form of a live culture, a frozen culture, an inoculum, or a lyophilised composition. Where provided in lyophilised form, the composition of the invention may comprise one or more lyobuffers, lyoprotectants, carriers, lubricants, preservatives, antioxidants, or the like. Lyophilised cultures may be accompanied by a nutrition source, e.g. in desiccated or liquid form, which may be mechanically associated with the lyophilized culture to facilitate mixing and stimulate growth.

In embodiments of the invention, the composition may comprise the engineered microorganism of the invention in an amount of at least about 1×10³ CFU/g, at least about 1×10⁵ CFU/g, at least about 1×10⁷ CFU/g, at least about 1×10⁸ CFU/g, at least about 1×10⁹ CFU/g or at least about 1×10¹⁰ CFU/g.

The engineered microorganism may be present as the sole strain present in the composition of the invention. Such compositions may comprise only de minimis or biologically irrelevant amounts of other bacterial strains or species.

Alternatively, the engineered microorganism may be present as part of a consortium, comprising i) 1 or more, 2 or more, 3 or more, 4 or more, 5 or more or 10 or more additional strains of microorganism and/or ii) 20 or fewer, 15 or fewer or 10 or fewer additional strains of microorganism. In certain embodiments, the composition of the invention may comprise the engineered microorganism of the invention and a second microorganism having the ability to produce C₂₋₄ alkanol or thiol such as ethanole, e.g. Saccharomyces cerevisiae.

The invention will now be described more specifically in the following examples.

The following examples are offered by way of illustration of certain embodiments of aspects of the application herein. None of the examples should be considered limiting on the scope of the application.

### Example 1

A strain of Saccharomyces cerevisiae (e.g. ATCC 96581) may be transformed to include the following three genes: methylthio-alkane reductase, MarBHDK and Cys, Met/SAM synthase, using a plasmid. Once transformed into the Saccharomyces cerevisiae strain, this engineered microorganism is anticipated to have the ability to convert ethanol to ethylene under anaerobic conditions and without the supply of oxygen, which ethylene can then be collected.

### Example 2: Cloning and Expression of an Oleate Hydratase (EC:4.2.1.53) and Linalool Dehydratase (EC:4.2.1.127) in Saccharomyces cerevisiae

### 1. Gene Synthesis, Polymerase Chain Reaction (PCR), and Purification of DNA Fragments

DNA sequences encoding an Oleate hydratase (EC:4.2.1.53) from Elizabethkingia meningoseptica (NCBI Taxonomy ID: 238—SEQ ID NO. 1) and Linalool dehydratase (EC:4.2.1.127) from Castellaniella defragrans (NCBI Taxonomy ID: 75697—SEQ ID NO. 2) were retrieved from the NCBI/Blast database. The obtained DNA sequences were subjected to codon optimization and addition of flanking regions using Geneious Prime® software.

Codon optimization was performed to enhance the expression efficiency of the target enzymes in Saccharomyces cerevisiae. Furthermore, specific flanking regions (≥35 DNA base pairs) were introduced to allow in vivo DNA assembly in Saccharomyces cerevisiae. The codon-optimised sequences comprising the flanking sequences are provided as SEQ ID NO. 3 and SEQ ID NO. 4, respectively. These flanking regions were incorporated into each of the codon-optimized gene sequences and in the regions downstream of the promoter and upstream of the terminator of the plasmids p416TEF1 and p416TPI1. Oligonucleotides were designed to introduce the same flanking regions (homologous) into the yeast plasmids p416TEF1 and p416TPI1. Oligonucleotides and genes were all synthesized by IDT Integrated DNA Technology™.

The p416TEF1 and p416TPI1 plasmids each contain elements for yeast replication, such as an origin of replication derived from yeast chromosomes, which allows the plasmid to replicate autonomously within the yeast cells. Additionally, each of the plasmids carries the selectable marker gene, URA3, which confers uracil prototrophy to yeast cells, enabling their growth and selection under specific conditions (such as growth in synthetic complete media without uracil). p416TEF1 contains the TEF (Translation Elongation Factor) promoter derived from the TEF1 gene while p416TPI1 contains the promoter TPI (Triose Phosphate Isomerase) derived from TPI1 gene. Both TEF1 and TPI1 promoters used here are derived from Saccharomyces cerevisiae and exhibit strong and constitutive expression activity. These promoters can drive robust and continuous transcription of the inserted gene of interest in yeast cells, ensuring high levels of protein production.

PCR amplification was performed to incorporate the flanking regions into each of the plasmids (p416TEF1 and p416TPI1) or to amplify the synthesized genes containing the flanking regions. All the PCR reactions were performed using PrimeSTAR® Max DNA Polymerase (R045B) from Takara Bio. Following PCR amplification, the gene fragments were purified using Monarch® PCR & DNA Cleanup Kit (T1030 L), while the linear plasmids were purified via gel excision using Zymoclean® Gel DNA Recovery Kit (D4007), according to the manufacturer's instructions.

### 2. Plasmid In Vivo DNA Assembly and Yeast Transformation

The purified linear PCR products (gene and plasmid), containing the flanking regions, were introduced into Saccharomyces cerevisiae cells (strain BY4741) using standard yeast transformation techniques (Gietz & Woods, 2006). The inclusion of the flanking regions in both the gene and plasmid allowed the precise assembly of these parts into a circular plasmid through homologous recombination in Saccharomyces cerevisiae cells.

After transformation, yeast cells were transferred to agar plates containing selective Synthetic Complete media with 2% glucose and lacking uracil. The plates were incubated at 30° C. for two days to allow the rise of individual colonies. Subsequently, 3 to 6 single colonies were transferred to new plates containing the same selective media for colony PCR screening.

### 3. Yeast Colony PCR Screening

Colony PCR analysis involved resuspending a single yeast colony in 20 microliters of a 20 millimolar NaOH solution. The cell suspension was subjected to a thermal incubation step at 99° C. for 15 minutes to achieve yeast cell lysis and DNA/plasmid exposure. Subsequently, PCR amplification was performed using the obtained DNA/NaOH solution and specific primers targeting the plasmid region encompassing the promoter and gene of interest.

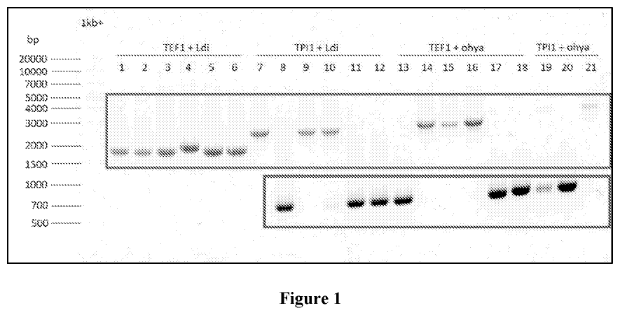

Colony PCR screening was performed using plasmid DNA from 21 different S. cerevisiae candidates and the results of this are shown in FIG. 1, which shows each of the 21 enumerated columns. DNA fragment size, shown on the left hand side of the FIGURE, was estimated using 1 kb+ DNA ladder. The lower box showing small DNA fragments (<1000 bp), correspond to incomplete constructions as the size of PCR products were smaller than expected. The upper box shows potentially correct candidates. All potentially correct candidates were sent for sequencing to confirm the presence and integrity of the promoter (TEF1 or TPI1) and genes. Gene for Linalool dehydratase (EC:4.2.1.127) is shown as “Ldi” and gene for Oleate hydratase (EC:4.2.1.53) as “ohya”. Plasmids p416TEF1 and p416TPI1 are shown as TEF1 and TPI1 respectively.

### Example 3: Sequence Optimisation for Expression in Chemoautolithotrophs

To demonstrate the versatility of the approaches detailed herein for use in engineering taxonomically and functionally different types of organisms, the genes having the sequences of SEQ ID NO. 1 and SEQ ID NO. 2, as well as genes encoding acyclic carotenoid 1,2-hydratase from Rubrivivax gelatinosus SEQ ID NO. 5 and carotenoid 1,2-hydratase (CruF) from Deinococcus radiodurans (ATCC 13939/DSM 20539) SEQ ID NO. 6 were optimized to render them suitable for expression in chemoautolithotrophic bacteria of different types. These include the denitrifyers Paracoccus denitrificans (SEQ IDS 7 to 10), Nitrospira japonica (SEQ IDS 11 to 14), and Nitrobacter winogradskyi (SEQ IDS 15 to 18), the iron oxidizers Acidithiobacillus ferrooxidans (SEQ IDS 19 to 22) and Ferroplasma acidiphilum (SEQ IDS 23 to 26) and the sulfur oxidizer Acidithiobacillus thiooxidans (SEQ IDS 27 to 30).

Optimization was performed for each organism by using tBLASTx to identify panels of genomic nucleotide sequences with the best identify to members from a panel of E. coli ribosomal proteins. Each organisms' sequence panel was then converted to CAI index using the python module CAI and used to optimize each dehydratase coding sequence.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. A microorganism engineered to express one or more enzymes which enhances the microorganism's ability to produce C₂₋₄ alkene, optionally ethylene from C₂₋₄ alkanol or thiol, optionally ethanol.

2. The microorganism of claim 1, wherein the microorganism is engineered to overexpress one or more enzymes capable of converting C₂₋₄ alkene, optionally ethylene from C₂₋₄ alkanol or thiol, optionally ethanol.

3. The microorganism of claim 1, wherein the microorganism is engineered to express one or more non-native enzymes capable of catalysing the conversion of C₂₋₄ alkanol or thiol, optionally ethanol to C₂₋₄ alkene, optionally ethylene.

4. The microorganism of claim 1, wherein the one or more enzymes are from the S-Adenosyl-L-methionine (SAM) pathway.

5. The microorganism of claim 1, wherein the one or more enzymes comprise methylthio-alkane reductase, MarBHDK and/or Cys, Met/SAM synthase.

6. The microorganism of claim 5, wherein the dehydratase enzyme utilises a cyclic substrate or an acyclic substrate.

7. The microorganism of claim 1, wherein the one or more enzymes which enhances the microorganism's ability to produce C₂₋₄ alkene do not utilise and/or produce oxygen.

8. The microorganism of claim 1, wherein the microorganism is engineered via plasmid transformation or manipulation of its genome.

9. The microorganism of claim 1, wherein the microorganism is an engineered version of a strain of:
the Rhodobacteriaceae family,
the Pseudomonaceae family,
the Blastochloridaceae family,
the Enterobacteriaceae family,
the Endomicrobaceae family,
the Acidithiobacillaceae family,
the Ferroplasmaceae family,
the Nitrospiraceae family, and/or
the Nitrobacteraceae family.

10. The microorganism of claim 1, wherein the microorganism is an engineered version of a fungal strain.

11. The microorganism of claim 10, wherein the fungal strain is one which produces C₂₋₄ alkanol or thiol, optionally ethanol.

12. The microorganism of claim 1, wherein the microorganism is anaerobic.

13. A process for producing C₂₋₄ alkene, optionally ethylene, comprising the step of:
i) contacting C₂₋₄ alkanol or thiol, optionally ethanol, with an enzyme capable of catalysing the conversion of C₂₋₄ alkanol or thiol, optionally ethanol to C₂₋₄ alkene, optionally ethylene, wherein the enzyme is expressed by a microorganism, and
ii) obtaining C₂₋₄ alkene, optionally ethylene.

14. The process of claim 13, wherein the microorganism is a microorganism engineered to express one or more enzymes which enhances the microorganism's ability to produce C₂₋₄ alkene, optionally ethylene from C₂₋₄ alkanol or thiol, optionally ethanol.

15. The process of claim 13, further comprising the step of
collecting the enzyme capable of catalysing the conversion of C₂₋₄ alkanol or thiol, optionally ethanol to C₂₋₄ alkene, optionally ethylene, from the microorganism, optionally prior to or simultaneous with step i).

16. The process of claim 13, wherein the process comprises contacting the microorganism with the C₂₋₄ alkanol or thiol, optionally ethanol in step i).

17. The process of claim 13, wherein the C₂₋₄ alkanol or thiol, optionally ethanol, is produced by the microorganism.

18. The process of claim 13, wherein the microorganism belongs to
the Rhodobacteriaceae family,
the Pseudomonaceae family,
the Blastochloridaceae family,
the Enterobacteriaceae family,
the Endomicrobaceae family,
the Acidithiobacillaceae family,
the Ferroplasmaceae family,
the Nitrospiraceae family, and/or
the Nitrobacteraceae family.

19. The process of claim 13, wherein the microorganism is a yeast.

20. The process of claim 13, wherein the microorganism is capable of producing C2-4 alkanol or thiol, optionally ethanol.

21. The process of claim 20, wherein the microorganism is engineered to enhance its ability to produce C2-4 alkanol or thiol, optionally ethanol.

22. The process of claim 13, wherein step i) of the process is operated in a reactor.

23. The process of claim 22, wherein the process comprises providing C2-4 alkanol or thiol, optionally ethanol in the reactor.

24. The process of claim 22, wherein the process comprises providing the microorganism in the reactor.

25. The process of claim 22, wherein the process comprises providing the enzyme in the reactor.

26. The process of claim 22, wherein the process comprises providing a nutrition source in the reactor.

27. The process of claim 26, wherein the nutrition source comprises a carbohydrate source, a nitrogen source and/or a phosphorous source.

28. The process of claim 13, wherein step i) is conducted at a temperature of about 20° C. to about 45° C.

29. The process of claim 13, wherein step i) is conducted at a temperature of about 35° C. to about 60° C.

30. The process of claim 13, wherein the step i) is conducted under anaerobic conditions.

31. The process of claim 30, wherein step i) is conducted under an atmosphere comprising oxygen at a level of about 10%.

32. The process of claim 30, wherein step i) is conducted under an atmosphere comprising oxygen at a level of about 5%.

33. A composition comprising the microorganism of claim 1, and a carrier.

34. The composition of claim 33, wherein the composition is in the form of a live culture, a frozen culture, an inoculum or a lyophilised composition.

35. The composition of claim 33, wherein the composition comprises the engineered microorganism in an amount of at least about 1×10⁵ CFU/g.

36. The composition of claim 33, wherein the engineered microorganism is present as the sole microorganism strain in the composition.

37. The composition of claim 33, wherein the composition comprises a second microorganism capable of producing C₂₋₄ alkanol or thiol, optionally ethanol.

38. The composition of claim 33, wherein the composition comprises the engineered microorganism as part of a consortium.
